(19) United States
(12) Patent Application Publication
(10) Pub. No.: US 2013/0109645 A1
(43) Pub. Date: May 2, 2013

(54) ADENOSINE RECEPTOR AGONISTS FOR THE TREATMENT AND PREVENTION OF VASCULAR OR JOINT CAPSULE CALCIFICATION DISORDERS

(71) Applicants: William A. Gahl, Kensington, MD (US); Manfred Boehm, Bethesda, MD (US); Cynthia St. Hilaire, Washington, DC (US); Shira G. Ziegler, Bethesda, MD (US); Thomas C. Markello, Rockville, MD (US)

(72) Inventors: William A. Gahl, Kensington, MD (US); Manfred Boehm, Bethesda, MD (US); Cynthia St. Hilaire, Washington, DC (US); Shira G. Ziegler, Bethesda, MD (US); Thomas C. Markello, Rockville, MD (US)

(73) Assignee: The united States of America,as represented by Secretary,Dept.,of Health and Human Services, Bethesda, MD (US)

(21) Appl. No.: 13/582,035

(22) PCT Filed: Mar. 30, 2011
(86) PCT No.: PCT/US2011/030493

§ 371 Date: Jan. 11, 2013

Related U.S. Application Data
(60) Provisional application No. 61/319,336, filed on Mar. 31, 2010.

(51) Int. Cl. A61K 31/7052; C07K 14/705; C12Q 1/44; C12Q 1/68; C07K 16/28; G01N 33/68

(52) U.S. Cl. CPC A61K 31/7052; C07K 16/2896; G01N 33/6893; C12Q 1/44; C12Q 1/686; C12Q 1/6827; C07K 14/70596
USPC 514/46; 536/23.5; 530/389.1; 435/7.21; 435/19; 435/320.1; 435/369; 514/341; 514/344; 435/6.11; 506/9; 530/350

(57) ABSTRACT

Disclosed are a method of treating or preventing a disorder in a mammal comprising administering to the mammal an adenosine receptor agonist or an adenosine receptor antagonist, either alone or in combination, in an amount effective to treat or prevent medial vascular or joint capsule calcification. Disclosed are methods of detecting or diagnosing a vascular or joint capsule calcification disorder, as well as a nucleic acid comprising a mutation in one or more exons of human NT5E selected from the group consisting of Exon 3, Exon 5, and Exon 9.

### CROSS-REFERENCE TO RELATED APPLICATION

This patent application claims the benefit of U.S. Provisional Patent Application No. 61/319,336, filed Mar. 31, 2010, which is incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 30,994 Byte ASCII (Text) file named “707797_ST25.txt,” dated Mar. 24, 2011.

### BACKGROUND OF THE INVENTION

Vascular or joint capsule calcification is characterized by the pathologic accumulation of calcium salts in vascular or joint tissues, respectively. The calcification of tissue can lead to hardening of the tissue and the onset of any of a variety of different disorders, many of which can be debilitating and painful.

Some of the disorders associated with vascular and/or joint capsule calcification include, for example, atherosclerosis, Monckeberg's medial sclerosis, Ehlers Danlos syndrome (EDS), Marfan/Loewe Dietz syndrome, fibromuscular dysplasia, Kawasaki syndrome, pseudoxanthoma elasticum, and premature placental calcification. Atherosclerosis is characterized by the accumulation of calcium-containing plaque inside the arteries, which can lead to a heart attack or stroke. Monckeberg's medial sclerosis is characterized by the formation of calcium deposits in the middle layer of the walls of vessels of patients suffering from diabetes mellitus or chronic renal disease. Ehlers-Danlos syndrome is a heterogeneous group of heritable connective tissue disorders, characterized by articular (joint) hypermobility, skin extensibility and tissue fragility. Loewe Dietz syndrome is a genetic disorder which affects blood vessels, particularly the aorta. Marfan syndrome is an inherited disorder of connective tissue. Fibromuscular dysplasia is a disorder that causes the narrowing of arteries in the kidneys, abdomen, as well as the carotid arteries. Kawasaki syndrome is largely seen in children under 5 years of age and causes inflammation in the walls of small- and medium-sized arteries throughout the body, including the coronary arteries. Pseudoxanthoma elasticum is characterized by the calcification of elastic fibers of connective tissue.

In spite of considerable research into diagnostics and therapies, there currently exists an unmet need for compositions and methods for detecting, treating, and/or preventing vascular or joint capsule calcification disorders.

### BRIEF SUMMARY OF THE INVENTION

An embodiment of the invention provides a method of treating or preventing medial vascular or joint capsule calcification in a mammal in need thereof, comprising administering to the mammal an adenosine receptor agonist in an amount effective to treat or prevent the medial vascular or joint capsule calcification.

Another embodiment of the invention provides a method of treating or preventing a disorder in a mammal in need thereof, comprising administering to the mammal an adenosine receptor agonist in an amount effective to treat or prevent the disorder, wherein the disorder is calciphylaxis, Monckeberg's medial sclerosis, arterial calcification due to deficiency of CD73 (ACDC), Ehlers Danlos syndrome, Marfan syndrome, Loewe Dietz syndrome, fibromuscular dysplasia, Kawasaki syndrome, pseudoxanthoma elasticum, or premature placental calcification.

Still another embodiment of the invention provides a method of treating or preventing vascular or joint capsule calcification in a mammal in need thereof, comprising administering to the mammal an adenosine receptor agonist in an amount effective to treat or prevent the vascular or joint capsule calcification, wherein the mammal has a NT5E mutation.

Another embodiment of the invention provides an adenosine receptor agonist for use in treating or preventing medial vascular or joint capsule calcification.

Another embodiment of the invention provides the use of an adenosine receptor agonist in the manufacture of a medicament for treating or preventing medial vascular or joint capsule calcification.

An embodiment of the invention provides a method of detecting or diagnosing a vascular or joint capsule calcification disorder comprising (a) obtaining a nucleic acid sample from a mammal; (b) obtaining a NT5E coding sequence from the nucleic acid sample; (c) comparing the NT5E coding sequence of the nucleic acid in the sample to a NT5E coding sequence of a negative control and obtaining a difference, wherein the difference is due to a mutation in the NT5E coding sequence of the nucleic acid in the sample; and (d) correlating the difference in the NT5E coding sequence of the nucleic acid in the sample as compared to the NT5E coding sequence of the negative control with a vascular or joint capsule calcification disorder.

Another embodiment of the invention provides a nucleic acid comprising a mutation in one or more exons of human NT5E selected from the group consisting of Exon 3, Exon 5, and Exon 9. The invention further provides embodiments including polypeptides, vectors, antibodies, and recombinant cells relating to the nucleic acids of the invention.

Still another embodiment of the invention provides a method of determining reduced activity or expression level of CD73 protein comprising: (a) determining the activity or expression level of CD73 protein of a biological sample from a mammal; (b) comparing the activity or expression level of the CD73 protein in the mammal with a negative control; and (c) correlating a decrease in activity or expression of the CD73 protein in the mammal as compared to the negative control with a vascular or joint capsule calcification disorder.

Another embodiment of the invention provides a method of determining an NT5E mutation comprising: (a) contacting a biological sample from a mammal with an antibody that binds to a polypeptide encoded by a nucleic acid comprising a mutation in Exon 5 or 9 of human NT5E but does not bind to a polypeptide encoded by non-mutant NT5E; and (b) detecting the binding of the antibody to the polypeptide encoded by a nucleic acid comprising a mutation in Exon 5 or 9 of human NT5E.

### BRIEF DESCRIPTION OF THE SEVERAL VIEWS OF THE DRAWING(S)

FIG. 1 is a graph showing intracellular levels of cyclic adenosine monophosphate (cAMP) (pmol/mL per ng protein) of control and arterial calcification due to deficiency of CD73 (ACDC) cells serum strayed overnight then given 30 μM exogenous AMP or 2 forskolin (positive control). N.T.=not treated.

FIG. 2 is a graph showing tissue-nonspecific alkaline phosphatase (TNAP) activity (nM pNP/μg protein) of control and ACDC cells that were cultured for 10 days and that were not treated (N.T.) or fed with osteogenic media alone (osteo), osteogenic media with dimethyl sulfoxide (DMSO) (vehicle), or with the A₁ agonist CCPA (10 μM) or the A₃ agonist IB-MECA (10 μM). **indicates p<0.005 using Student's t-test.

### DETAILED DESCRIPTION OF THE INVENTION

An embodiment of the invention provides a method of treating or preventing medial vascular or joint capsule calcification in a mammal in need thereof, comprising administering to the mammal an adenosine receptor agonist or an adenosine receptor antagonist, either alone or in combination, in an amount effective to treat or prevent the medial vascular or joint capsule calcification.

An embodiment of the present invention is predicated on the discovery of a mutation in the NT5E gene that leads to vascular and/or joint capsule calcification. NT5E (5′-nucleotidase, ecto) encodes cluster of differentiation (CD) 73, an enzyme that converts extracellular adenosine monophosphate (AMP) to adenosine and inorganic phosphate. NT5E mutations result in CD73 deficiency which, in turn, leads to a decrease in adenosine and, ultimately, an increase in calcification, particularly, medial vascular and joint capsule calcification. In particular, CD73 deficiency may lead to Arterial Calcification due to Deficiency of CD73 (ACDC), which may be characterized by medial vascular and joint capsule calcification.

Extracellular adenosine binds to four subtypes of adenosine receptors, namely, A₁, A_{2A}, A_{2B}, and A₃. The binding of adenosine to A₁ and A₃ receptors decreases the production of cyclic AMP (cAMP). Without being bound to a particular theory or mechanism, it is believed that an increase in cAMP levels leads to vascular and/or joint capsule calcification. Accordingly, it is believed that A₁ and A₃ agonists decrease cAMP levels and, therefore, decrease vascular and/or joint capsule calcification. Conversely, the binding of adenosine to A_{2A} and A_{2B} receptors stimulates the production of cAMP. Without being bound to a particular theory or mechanism, it is believed that A_{2A} and A_{2B} agonists increase cAMP levels and, therefore, decrease vascular and/or joint capsule calcification. Without being bound to a particular theory or mechanism, it is believed that the lack of adenosine receptor signaling results in medial vascular calcification. Thus, an adenosine receptor agonist modulates tissue-nonspecific alkaline phosphatase (TNAP) expression and activity and is believed to treat or prevent medial vascular and/or joint capsule calcification.

An embodiment of the invention provides a method of treating or preventing medial vascular or joint capsule calcification in a mammal in need thereof, comprising administering to the mammal an adenosine receptor agonist in an amount effective to treat or prevent the medial vascular or joint capsule calcification. In an embodiment of the invention, the vascular calcification is medial vascular calcification.

Another embodiment of the invention provides a method of treating or preventing a disorder in a mammal in need thereof, comprising administering to the mammal an adenosine receptor agonist in an amount effective to treat or prevent the disorder, wherein the disorder is atherosclerosis, calciphylaxis, Monckeberg's medial sclerosis, ACDC, Ehlers Danlos syndrome, Marfan syndrome, Loewe Dietz syndrome, fibromuscular dysplasia, Kawasaki syndrome, pseudoxanthoma elasticum, or premature placental calcification. In an embodiment, the disorder does not include atherosclerosis.

In an embodiment, the adenosine receptor agonist is an A₁ adenosine receptor agonist. The A₁ adenosine receptor agonist may be any suitable pharmaceutically active agent agonist for the A₁ adenosine receptor. In an embodiment, the A₁ adenosine receptor agonist is a compound of general formula (I); see, for example, formula (I) at page 4, line 1 to page 6, line 23 of World Inellectual Property Organization Publication No. 2000/40251, which is incorporated herein by reference:

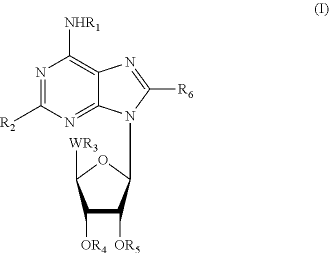

wherein R₁ represents a lower alkyl (e.g., C₁-C₃), substituted or unsubstituted cycloalkyl (e.g., C₃-C₈); a hydroxyl or hydroxyalkyl; a phenyl, anilide, or lower alkyl phenyl, all optionally substituted by one or more substituents —SOR^{c}, —SO₂R^{c}, —SO₃H, —SO₂NR^{a}R^{b}, —OR^{a}, —SR^{a}, —NHSO₂R^{c}, —NHCOR^{a}, —NR^{a}R^{b}, or —NHR^{a}CO₂R^{b}; wherein

R^{a} and R^{b} represent independently a hydrogen, lower alkyl, alkanoyl, amine, phenyl or naphthyl, the alkyl group optionally being substituted with a substituted or unsubstituted phenyl or phenoxy group; or when R₁ represents —NR^{a}R^{b}, said R^{a} and R^{b} form together with the nitrogen atom a 5- or 6-membered heterocyclic ring optionally containing a second heteroatom selected from oxygen or nitrogen, which second nitrogen heteroatom may optionally be further substituted by hydrogen or lower alkyl; or —NR^{a}R^{b} is a group of general formula (II) or (III):

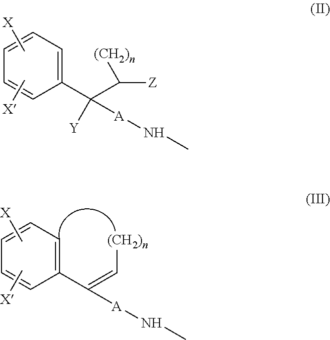

wherein n is an integer from 1 to 4; Z is hydrogen, lower alkyl (e.g., C₁-C₃) or hydroxyl; Y is hydrogen, lower alkyl, or OR′ where R′ is hydrogen, lower alkyl or lower alkanoyl (e.g., C₁-C₃); A is a bond or a lower alkylene; X and X′ are each independently hydrogen, lower alkyl (e.g., C₁-C₃), lower alkoxy, hydroxy, lower alkanoyl, nitro, haloalkyl such as trifluoromethyl, halogen, amino, mono- or di-lower alkyl amino, or when X and X′ are taken together a methylenedioxy group;

R^{c} represents a lower alkyl (e.g., C₁-C₃); or

R₁ represents an epoxide substitutent of general formulae (IVa) or (IVb):

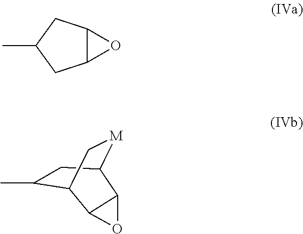

wherein M is a lower alkyl group (e.g., C₁-C₃);

R₂ represents hydrogen; halogen; substituted or unsubstituted lower alkyl or alkenyl group; lower haloalkyl or alkenyl; cyano; acetoamido; lower alkoxy; lower alkylamino; NR^{d}R^{e} where R^{d} and R^{e} are independently hydrogen, lower alkyl, phenyl or phenyl substituted by lower alkyl, lower alkoxy, halogen or haloalkyl or alkoxyl; —SR^{f} where R^{f} is hydrogen, lower alkyl, lower alkanoyl, benzoyl or phenyl;

W represents the group —OCH₂—, —NHCH₂—, —SCH₂—, or —NH(C═O)—;

R₃, R₄ and R₅ represent independently a hydrogen, lower alkyl or lower alkenyl, branched or unbranched C₁-C₁₂ alkanoyl, benzoyl or benzoyl substituted by lower alkyl, lower alkoxy, halogen, or R₄ and R₅ form together a 5-membered ring optionally substituted by a lower alkyl or alkenyl; R₃ further represents independently a phosphate, hydrogen or dihydrogen phosphate, or an alkali metal or ammonium or dialkali or diammonium salt thereof;

R₆ represents a hydrogen or halogen atom; or one of the substituents R₁ to R₆ is a sulfohydrocarbon radical (e.g., sulfonate) of the formula R^{g}—SO₃—R^{h}—, wherein R^{g} represents a group selected from C₁-C₁₀ aliphatic, phenyl and lower alkyl substituted aromatic group which may be substituted or unsubstituted and R^{h} represents a monovalent cation, and the non-sulfur containing substituents being as defined above; or isomers, diastereomers, pharmaceutically acceptable salts or solvates of said compound.

In accordance with an embodiment of the invention, the A₁ adenosine receptor agonist is selected from the group consisting of CPA (N⁶-cyclopentyladenosine); CCPA (2-chloro-N(6)-cyclopentyladenosine); S(−)-ENBA ((2S)—N⁶-(2-endo-norbornyl)adenosine); ADAC(N⁶-[4-[[[4-[[[(2-aminoethyl)amino]carbonyl]methyl]-anilino]carbonyl]methyl]phenyl]adenosine); AMP579; NNC-21-0136 (2-chloro-N-[(R)-[(2-benzothiazoly)thio]-2-propyl]adenosine); GR79236; CVT-510 (also known as tecadenoson) ((2R,3S,4R,5R)-2-(hydroxymethyl)-5-[6-[[(3S)-oxolan-3-yl]amino]purin-9-yl]oxolane-3,4-diol; CVT-2759 (5′-O—(N-methylcarbamoyl)-N6-[tetrahydrofuran-3(R)-yl]adenosine); SDZ WAG 994 (N-cyclohexyl-2′-O-methyladenosine); and selodenoson (also known as RG 14202) ((2S,3S,4R,5R)-5-[6-(cyclopentylamino)purin-9-yl]-N-ethyl-3,4-dihydroxyoxolane-2-carboxamide).

CPA has Chemical Structure (1):

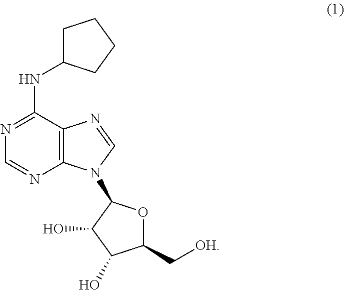 [SMILES: OC[C@@H]1O[C@H](n2cnc3c(NC4CCCC4)ncnc32)[C@@H](O)[C@H]1O]

CCPA has Chemical Structure (2):

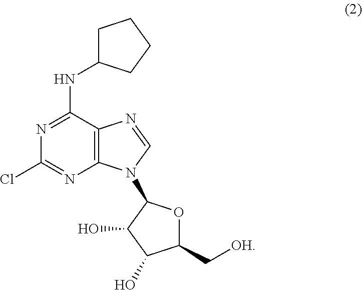 [SMILES: OC[C@@H]1O[C@H](n2cnc3c(NC4CCCC4)nc(Cl)nc32)[C@@H](O)[C@H]1O]

S(−)-ENBA has Chemical Structure (3):

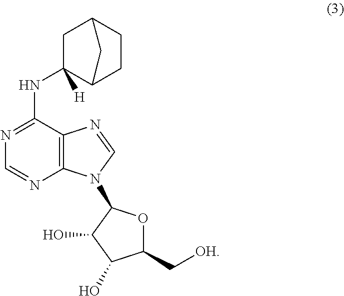 [SMILES: OC[C@@H]1O[C@H](n2cnc3c(N[C@@H]4CC5CCC4C5)ncnc32)[C@@H](O)[C@H]1O]

ADAC has Chemical Structure (4):

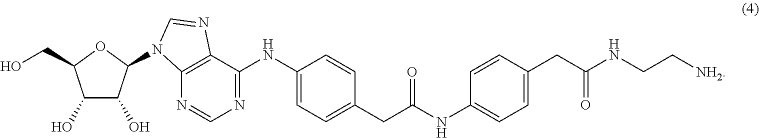 [SMILES: NCCNC(=O)Cc1ccc(NC(=O)Cc2ccc(Nc3ncnc4c3ncn4[C@@H]3O[C@H](CO)[C@@H](O)[C@H]3O)cc2)cc1]

AMP579 has Chemical Structure (5):

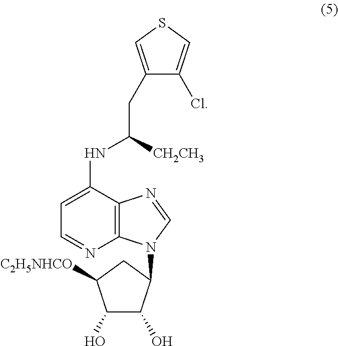

NNC-21-0136 has Chemical Structure (6):

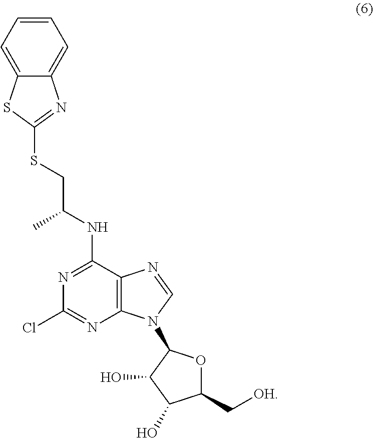 [SMILES: C[C@H](CSc1nc2ccccc2s1)Nc1nc(Cl)nc2c1ncn2[C@H]1O[C@@H](CO)[C@H](O)[C@@H]1O]

GR79236 has Chemical Structure (7):

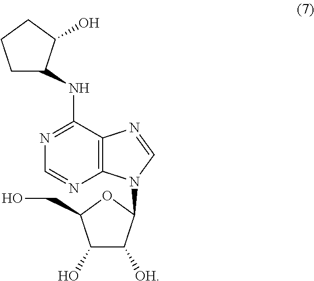

CVT-510 (tecadenoson) has Chemical Structure (8):

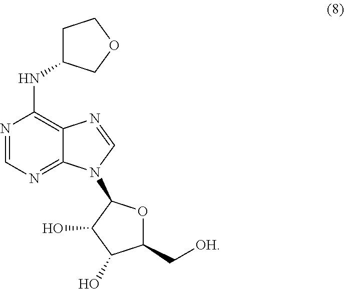 [SMILES: OC[C@@H]1O[C@H](n2cnc3c(N[C@@H]4CCOC4)ncnc32)[C@@H](O)[C@H]1O]

CVT-2759 has Chemical Structure (9):

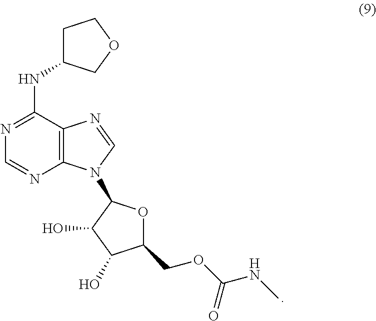 [SMILES: CNC(=O)OC[C@@H]1O[C@H](n2cnc3c(N[C@@H]4CCOC4)ncnc32)[C@@H](O)[C@H]1O]

SDZ WAG 994 has Chemical Structure (10):

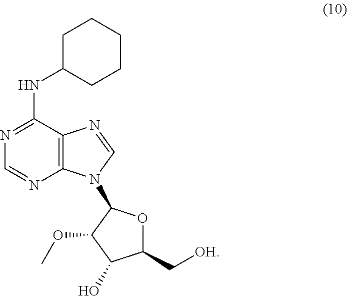 [SMILES: CO[C@H]1[C@@H](O)[C@H](CO)O[C@@H]1n1cnc2c(NC3CCCCC3)ncnc21]

Selodenoson (RG 14202) has Chemical Structure (11):

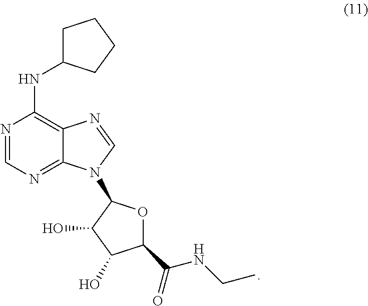 [SMILES: CCNC(=O)[C@@H]1O[C@H](n2cnc3c(NC4CCCC4)ncnc32)[C@@H](O)[C@H]1O]

In an embodiment, the adenosine receptor agonist is an A₃ adenosine receptor agonist. The A₃ adenosine receptor agonist may be any suitable pharmaceutically active agent agonist for the A₃ adenosine receptor; see, for example, formula (I) at column 6, line 65 to column 8, line 25 of U.S. Pat. No. 7,064,112, which is incorporated herein by reference. In an embodiment, the A₃ adenosine receptor agonist is a compound of general formula (V):

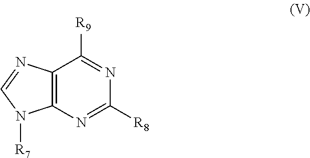

wherein R₇ is C₁-C₁₀ alkyl, C₁-C₁₀ hydroxyalkyl, C₁-C₁₀ carboxyalkyl or C₁-C₁₀ cyanoalkyl or a group of the following general formula (VI):

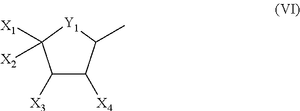

in which:

Y₁ is an oxygen or sulfur atom or CH₂;

X₁ is H, C₁-C₁₀ alkyl, R^{100a}R^{100b}NC(═O)— or HOR^{100c}—, wherein R^{100a} and R^{100b} may be the same or different and are selected from the group consisting of hydrogen, C₁-C₁₀ alkyl, amino, C₁-C₁₀ haloalkyl, C₁-C₁₀ aminoalkyl, C₁-C₁₀ t-butoxycarbonyl-aminoalkyl, and C₃-C₁₀ cycloalkyl or are joined together to form a heterocyclic ring containing two to five carbon atoms, and R^{100c} is selected from the group consisting of C₁-C₁₀ alkyl, amino, C₁-C₁₀ haloalkyl, C₁-C₁₀ aminoalkyl, C₁-C₁₀ butyloxycarbonyl (BOC)-aminoalkyl, and C₃-C₁₀ cycloalkyl;

X₂ is H, hydroxyl, C₁-C₁₀ alkylamino, C₁-C₁₀ alkylamido or C₁-C₁₀ hydroxyalkyl;

X₃ and X₄ each independently are hydrogen, hydroxyl, amino, amido, azido, halo, alkyl, alkoxy, carboxy, nitrilo, nitro, trifluoro, aryl, alkaryl, mercapto, thioester, thioether, —OCOPh, —OC(═S)OPh or both X₃ and X₄ are oxygen connected to >C═S to form a 5-membered ring, or X₂ and X₃ form the ring of foimula (VII):

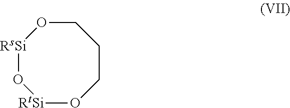

where R^{s} and R^{t} are independently C₁-C₁₀ alkyl;

R₈ is selected from the group consisting of hydrogen, halo, C₁-C₁₀ alkylether, amino, hydrazido, C₁-C₁₀ alkylamino, C₁-C₁₀ alkoxy, C₁-C₁₀ thioalkoxy, pyridylthio, C₂-C₁₀ alkenyl, C₂-C₁₀ alkynyl, mercapto, and C₁-C₁₀ alkylthio; and

R₉ is a —NR₁₀R₁₁ group with R₁₀ being hydrogen, alkyl, substituted alkyl or aryl-NH—C(Z₁)—, with Z₁ being O, S or NR^{100a}, and, when R₁₀ is hydrogen, R₁₁ being selected from the group consisting of R- and S-1-phenylethyl, benzyl, phenylethyl or anilide groups, each said group being unsubstituted or substituted in one or more positions with a substituent selected from the group consisting of C₁-C₁₀ alkyl, amino, halo, C₁-C₁₀ haloalkyl, nitro, hydroxyl, acetamido, C₁-C₁₀ alkoxy, and sulfonic acid or a salt thereof; or R₁₁ being benzodioxanemethyl, furfuryl, L-propylalanylaminobenzyl, β-alanylaminobenzyl, t-BOC-β-alanylaminobenzyl, phenylamino, carbamoyl, phenoxy or C₁-C₁₀ cycloalkyl; or R₁₁ being a group of the following formula (VIII):

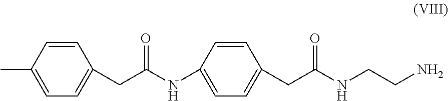 [SMILES: Cc1ccc(CC(=O)Nc2ccc(CC(=O)NCCN)cc2)cc1]

or, when R₁₀ is alkyl, substituted alkyl, or aryl-NH—C(Z₁)—, then R₁₁ being selected from the group consisting of substituted or unsubstituted heteroaryl-NR^{100a}—C(Z₁), heteroaryl-C(Z₁)—, alkaryl-NR^{100a}—C(Z₁)—, alkaryl-C(Z₁)—, aryl-NR—C(Z₁)— and aryl-C(Z₁); or isomers, diastereomers, pharmaceutically acceptable salts or solvates of said compound.

In accordance with an embodiment of the invention, the A₃ adenosine receptor agonist is selected from the group consisting of IB-MECA ((2S,3S,4R,5R)-3,4-dihydroxy-5-[6-[(3-iodophenyl)methylamino]purin-9-yl]-N-methyloxolane-2-carboxamide); C1-IB-MECA; LJ568; CP-608039; MRS3558 ((1′S,2′R,3′S,4′R,5′S)-4-(2-chloro-6-(3-chlorobenzylamino)-9H-purin-9-yl)-2,3-dihydroxy-N-methylbicyclo [3.1.0] hexane-1-carboxamide); and MRS1898.

IB-MECA has Chemical Structure (12):

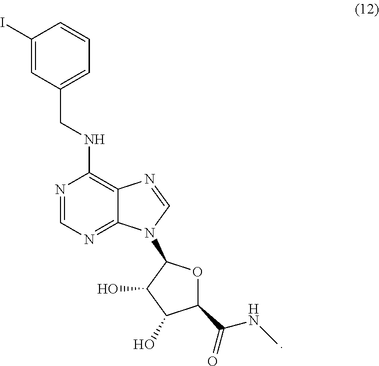 [SMILES: CNC(=O)[C@@H]1O[C@H](n2cnc3c(NCc4cccc(I)c4)ncnc32)[C@@H](O)[C@H]1O]

C1-IB-MECA has Chemical Structure (13):

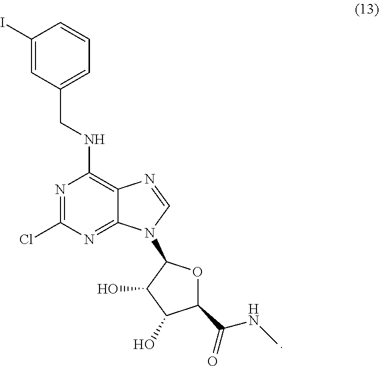 [SMILES: CNC(=O)[C@@H]1O[C@H](n2cnc3c(NCc4cccc(I)c4)nc(Cl)nc32)[C@@H](O)[C@H]1O]

LJ568 has Chemical Structure (14):

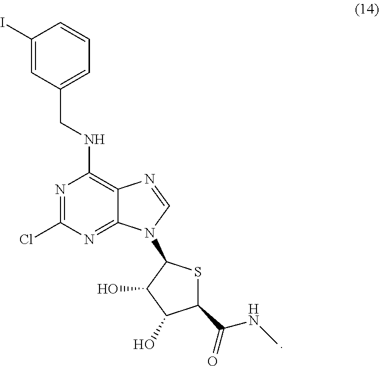 [SMILES: CNC(=O)[C@@H]1S[C@H](n2cnc3c(NCc4cccc(I)c4)nc(Cl)nc32)[C@@H](O)[C@H]1O]

CP-608039 has Chemical Structure (15):

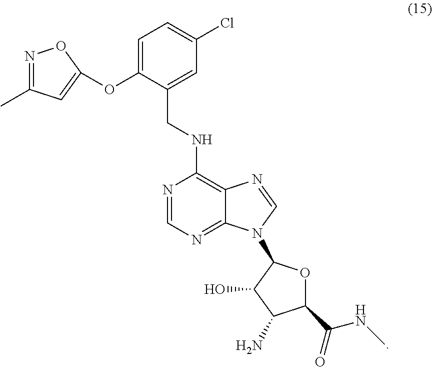

MRS3558 has Chemical Structure (16):

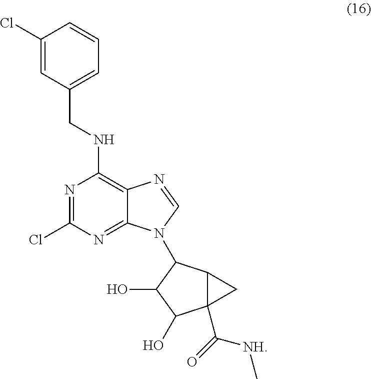 [SMILES: CNC(=O)C12CC1C(n1cnc3c(NCc4cccc(Cl)c4)nc(Cl)nc31)C(O)C2O]

MRS1898 has Chemical Structure (17):

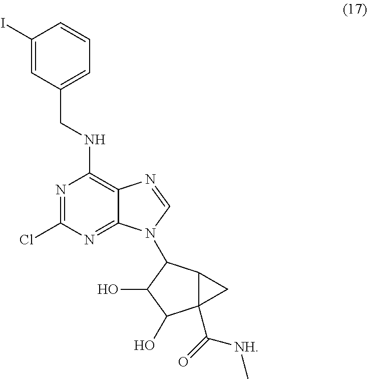 [SMILES: CNC(=O)C12CC1C(n1cnc3c(NCc4cccc(I)c4)nc(Cl)nc31)C(O)C2O]

In an embodiment, the adenosine receptor agonist is an A_{2A} adenosine receptor agonist. The A_{2A} adenosine receptor agonist may be any suitable pharmaceutically active agent agonist for the A_{2A} adenosine receptor. In an embodiment, the A_{2A} adenosine receptor agonist is a compound of general formula (IX); see, for example, the formula at column 3, line 10 to column 4, line 56 of U.S. Pat. No. 7,655,637, which is incorporated herein by reference:

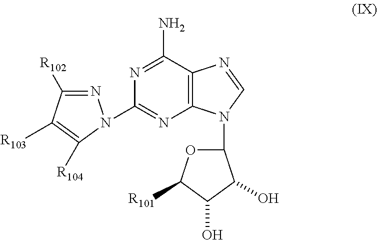

wherein R₁₀₁═CH₂OH or —CONR₁₀₅R₁₀₆;

R₁₀₃ is independently selected from the group consisting of C₁₋₁₅ alkyl, halo, NO₂, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂, —CONR₁₀₇R₁₀₈, C₂₋₁₅ alkenyl, C₂₋₁₅ alkynyl, heterocyclyl, aryl, and heteroaryl, wherein the alkyl, alkenyl, alkynyl, aryl, heterocyclyl and heteroaryl substituents are optionally substituted with from 1 to 3 substituents independently selected from the group consisting of halo, alkyl, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)_{2 NR}²⁰COR²², NR²⁰CO₂R²², NR²⁰CON(R²⁰)₂, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein the optionally substituted heteroaryl, aryl, and heterocyclyl substituents are optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, or OR²⁰;

R₁₀₅ and R₁₀₆ are each individually selected from H, and C₁-C₁₅ alkyl that is optionally substituted with from 1 to 2 substituents independently selected from the group of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ wherein each optionally substituted heteroaryl, aryl, and heterocyclyl substituent is optionally substituted with halo, NO₂, alkyl, CF₃, amino, monoalkylamino, dialkylamino, alkylamide, arylamide, heteroarylamide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, and OR²⁰;

R₁₀₇ is selected from the group consisting of hydrogen, C₁₋₁₅ alkyl, C₂₋₁₅ alkenyl, C₂₋₁₅ alkynyl, heterocyclyl, aryl and heteroaryl, wherein the alkyl, alkenyl, alkynyl, aryl, heterocyclyl and heteroaryl substituents are optionally substituted with from 1 to 3 substituents independently selected from the group of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰ and OCON(R²⁰)₂ and wherein each optionally substituted heteroaryl, aryl and heterocyclyl substituent is optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N (R²⁰)₂, SR²⁰, S(O)R²², SO₂R²², SO₂N(R²⁰)₂, CN, and OR²⁰;

R₁₀₈ is selected from the group consisting of hydrogen, C₁₋₁₅ alkyl, C₂₋₁₅ alkenyl, C₂₋₁₅ alkynyl, heterocyclyl, aryl, and heteroaryl, wherein the alkyl, alkenyl, alkynyl, aryl, heterocyclyl, and heteroaryl substituents are optionally substituted with from 1 to 3 substituents independently selected from the group consisting of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein each optionally substituted heteroaryl, aryl, and heterocyclyl substituent is optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, and OR²⁰;

R²⁰ is selected from the group consisting of H, C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl substituents are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl;

R²² is selected from the group consisting of C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl substituents are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl; and

wherein R₁₀₂ and R₁₀₄ are selected from the group consisting of H, C₁₋₆ alkyl and aryl, wherein the alkyl and aryl substituents are optionally substituted with halo, CN, CF₃, OR²⁰ and N(R²⁰)₂ with the proviso that when R₁₀₂ is not hydrogen then R₁₀₄ is hydrogen, and when R₁₀₄ is not hydrogen then R₁₀₂ is hydrogen.

In accordance with an embodiment of the invention, the A_{2A} adenosine receptor agonist is selected from the group consisting of NECA (5′-N-ethylcarboxamido adenosine); CGS21680 (2-p-(2-Carboxyethyl)phenethylamino-5′-N-ethylcarboxamidoadenosine); MRE-0094 (2-[2-(4-chlorophenyl)ethoxy]adenosine); DPMA (N⁶-[2-(3,5-dimethoxyphenyl)-2-(2-methylphenyl)ethyl]adenosine); Glaxo compound; binodenoson (also known as MRE0740) (2-(cyclohexylmethylidenehydrazino)adenosine); apadenoson (also known as ATL-146e); ATL-313; and Regadenoson (also known as CV-3146) (1-[6-amino-9-[(2R,3R,4S,5R)-3, 4-dihydroxy-5-(hydroxymethyl)oxolan-2-yl]purin-2-yl]-N-methylpyrazole-4-carboxamide).

NECA has Chemical Structure (30):

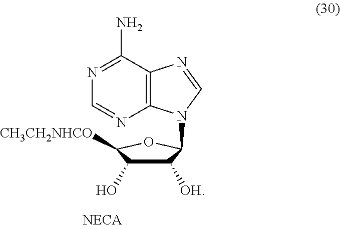

CGS21680 has Chemical Structure (31):

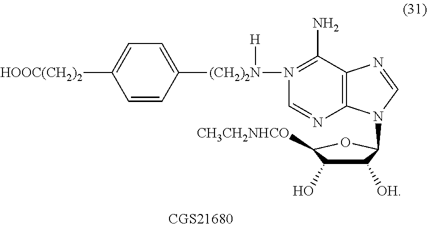

MRE-0094 has Chemical Structure (32):

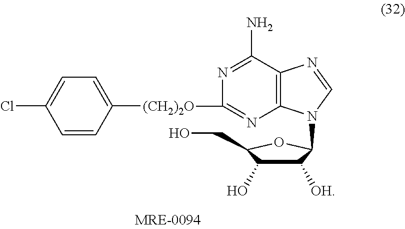

DPMA has Chemical Structure (33):

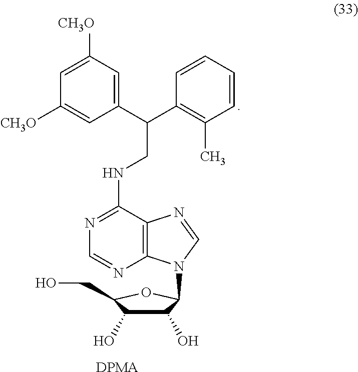

Glaxo Compound has Chemical Structure (34):

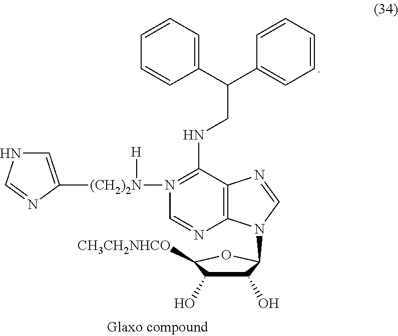

Binodenoson (MRE0740) has Chemical Structure (35):

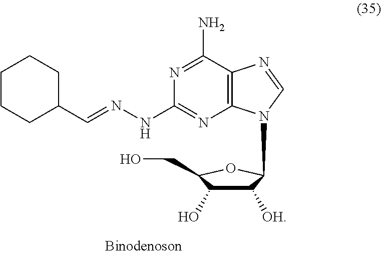

Apadenoson (ATL-146e) has Chemical Structure (36):

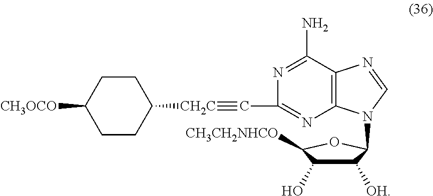

ATL-313 has Chemical Structure (37):

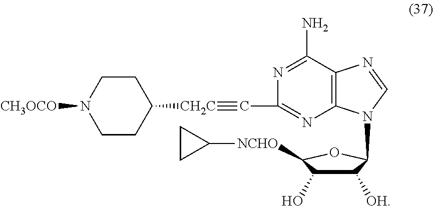

Regadenoson (CV-3146) has Chemical Structure (38):

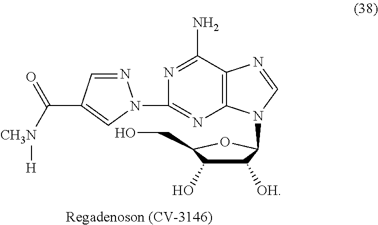

In an embodiment, the adenosine receptor agonist is an A_{2B} adenosine receptor agonist. The A_{2B} adenosine receptor agonist may be any suitable pharmaceutically active agent agonist for the A_{2B} adenosine receptor. In an embodiment, the A_{2B} adenosine receptor agonist is a compound of general formula (X); see, for example, formula (I) at paragraphs [0009]-[0013] of U.S. Patent Application Publication No. 2009/0221649 which is incorporated herein by reference:

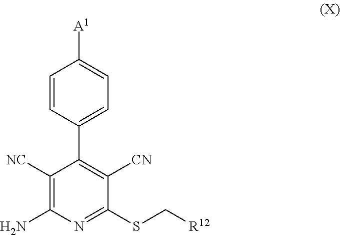

wherein

A¹ represents —O—R¹³ or —NH—C(═O)—R¹⁴;

R¹² represents CH₁₂—C(═O)—NH₂, pyridyl or thiazolyl;

R¹³ represents hydrogen or (C₃-C₆)-cycloalkylmethyl, and

R¹⁴ represents (C₁-C₄)-alkyl, (C₁-C₄)-alkoxy, mono- or di-(C₁-C₄)-alkylamino.

In accordance with an embodiment of the invention, the A_{2B} adenosine receptor agonist is selected from the group consisting of LUF5835 and Bay-60-6583.

LUF5835 has Chemical Structure (39):

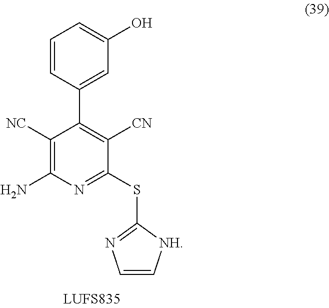 [SMILES: N#Cc1c(N)nc(Sc2ncc[nH]2)c(C#N)c1-c1cccc(O)c1]

Bay-60-6583 has Chemical Structure (40):

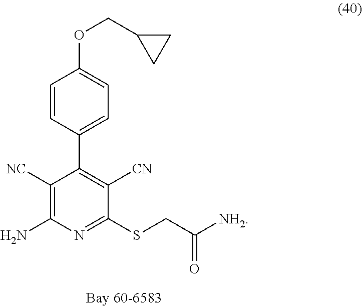 [SMILES: N#Cc1c(N)nc(SCC(N)=O)c(C#N)c1-c1ccc(OCC2CC2)cc1]

An embodiment of the invention provides a method of treating or preventing medial vascular or joint capsule calcification in a mammal in need thereof, comprising administering to the mammal an adenosine receptor agonist or an adenosine receptor antagonist, either alone or in combination, in an amount effective to treat or prevent the medial vascular or joint capsule calcification.

In an embodiment, the adenosine receptor antagonist is an A₁ adenosine receptor antagonist. The A₁ adenosine receptor antagonist may be any suitable pharmaceutically active agent antagonist for the A₁ adenosine receptor. In an embodiment, the A₁ adenosine receptor antagonist is a compound of general formula (XI); see, for example, the formula at page 1, paragraphs [0007]-[0011] of U.S. Patent Application Publication No. 2005/0059683, which is incorporated herein by reference:

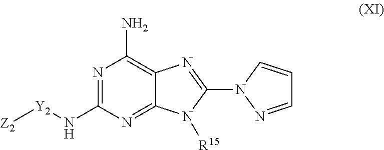

wherein:

R₁₅ is optionally substituted C₁₋₄ alkyl;

Y₂ is C₁₋₄ alkylene; and

Z₂ is phenyl that is optionally substituted with halo, hydroxy, amino, cyano, or optionally substituted C₁₋₄ alkyl.

In accordance with an embodiment of the invention, the A₁ adenosine receptor antagonist is selected from the group consisting of DPCPX (8-Cyclopentyl-1,3-dipropylxanthine); BG 9928 (also known as BIO-9002) (1,3-dipropyl-8-[1-(4-propionate)-bicyclo-[2,2,2]octyl]xanthine); N-0861 (N6-endonorbornyl-9-methyladenine); WRC-0571 (8-(N-methylisopropyl)amino-N6-(5′-endohydroxy-endonorbornyl)-9-methyladenine); FK 453 (6-Oxo-3-(2-phenylpyrazolo[1,5-a]pyridin-3-yl)-1(6H)-pyridazinebutanoic acid); FR194921; BG 9719 (also known as CVT-124) (1,3-dipropyl-8-[2-(5,6-epoxynorbornyl)]xanthine); and KW3902 (8-(noradamantan-3-yl)-1,3-dipropylxanthine).

DPCPX has Chemical Structure (41):

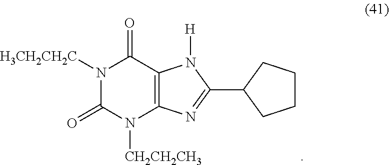

BG 9928 has Chemical Structure (42):

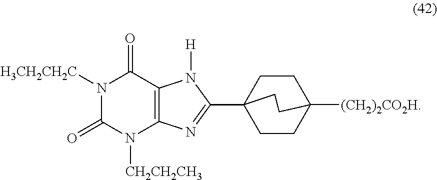

N-0861 has Chemical Structure (43):

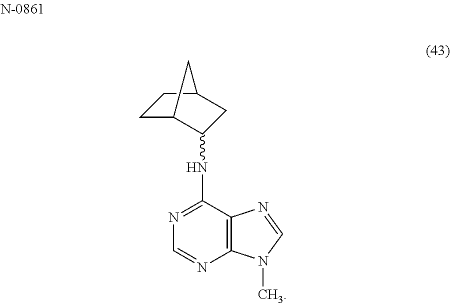 [SMILES: Cn1cnc2c(NC3CC4CCC3C4)ncnc21]

WRC-0571 has Chemical Structure (44):

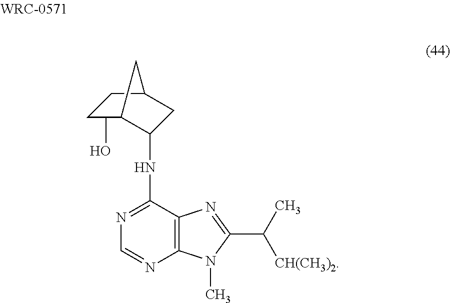 [SMILES: CC(C)C(C)c1nc2c(NC3CC4CCC(O)C3C4)ncnc2n1C]

FK 453 has Chemical Structure (45):

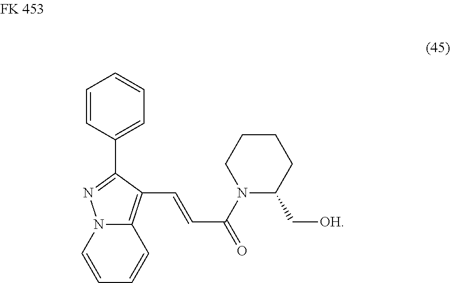 [SMILES: O=C(/C=C/c1c(-c2ccccc2)nn2ccccc12)N1CCCC[C@@H]1CO]

FR194921 has Chemical Structure (46):

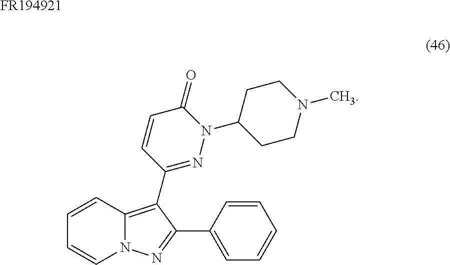 [SMILES: "FR194921" ; CN1CCC(n2nc(-c3c(-c4ccccc4)nn4ccccc34)ccc2=O)CC1]

BG 9719 has Chemical Structure (47):

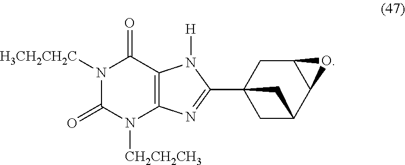

KW3902 has Chemical Structure (48):

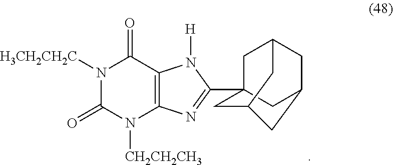

In an embodiment, the adenosine receptor antagonist is an A₃ adenosine receptor antagonist. The A₃ adenosine receptor antagonist may be any suitable pharmaceutically active agent antagonist for the A₃ adenosine receptor. In an embodiment, the A₃ adenosine receptor antagonist is a compound of general formula (XII); see, for example, formula (I) at column 4, lines 10-38 of U.S. Pat. No. 6,376,521, which is incorporated herein by reference:

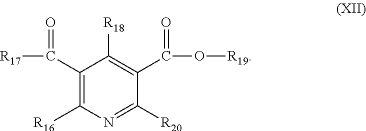

wherein R₁₆ is selected from the group consisting of C₁-C₆ alkyl, C₃-C₇ cycloalkyl, and C₁-C₆ alkoxy C₁-C₆ alkyl; R₁₇ is selected from the group consisting of C₁-C₆ alkoxy, C₁-C₆ alkylsulfanyl, hydroxy, C₁-C₆ alkoxy C₁-C₆ alkylsulfanyl, hydroxy C₁-C₆ alkylsulfanyl, and halo C₁-C₆ alkylsulfanyl, or R₁₇ together with R₁₈ forms a 3-7 membered heterocyclic ring containing O, N, or S; R₁₈ is selected from the group consisting of C₁-C₆ alkyl, halo C₁-C₆ alkyl, hydroxy C₁-C₆ alkyl, C₁-C₆ alkoxy, C₁-C₆ alkysulfanyl, C₁-C₆ alkylamino, C₁-C₆ alkylcarbonyl sulfanyl C₁-C₆ alkyl, aryl C₂-C₆ alkenyl, aryl C₂-C₆ alkynyl, formyl, and acetal; R₁₉ is selected from the group consisting of C₁-C₆ alkyl, aryl C₁-C₆ alkyl, hydroxy C₁-C₆ alkyl, and halo C₁-C₆ alkyl; and R₂₀ is selected from the group consisting of aryl, C₃-C₇ cycloalkyl, and haloaryl; wherein the aryl is a phenyl or naphthyl. The nitrogen atom of the heterocyclic ring can be saturated or unsaturated: Thus, for example, the heterocyclic ring can contain an NH group or an NR₂₁ group wherein R₂₁ is a C₁-C₆ alkyl, aryl, formyl, or C₁-C₆ acyl.

In accordance with an embodiment of the invention, the A₃ adenosine receptor antagonist is selected from the group consisting of OT-7999; MRS1292; VUF5574 (N-(2-methoxyphenyl)-N′-[2-(3-pyridinyl)-4-quinazolinyl]-urea); PSB-11; MRS3777 (2-phenoxy-6-(cyclohexylamino)purine hemioxalate); MRS1334 (1,4-dihydro-2-methyl-6-phenyl-4-(phenylethynyl)-3,5-pyridinedicarboxylic acid 3-ethyl-5-[(3-nitrophenyl)-methyl]ester); FA385; MRE 3008-F20 (5-[[(4-methoxyphenyl)amino]carbonyl]amino-8-ethyl-2-(2-furyl)-pyrazolo[4,3-e]1,2,4-triazolo[1,5-c]pyrimidine); MRS1523 (2,3-diethyl-4,5-dipropyl-6-phenylpyridine-3-thiocarboxylate-5-carboxylate); Novartis compound; and MRS1220 (9-chloro-2-(2-furanyl)-5-[(phenylacetyl)amino][1,2,4]-triazolo[1,5-c]quinazoline).

OT-7999 has Chemical Structure (49):

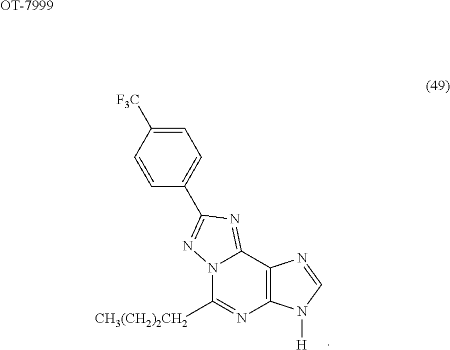

MRS1292 has Chemical Structure (50):

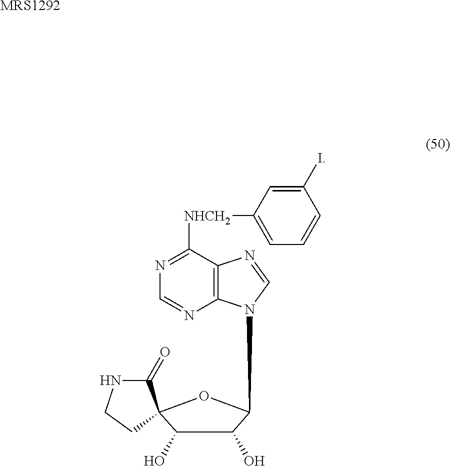

VUF5574 has Chemical Structure (51):

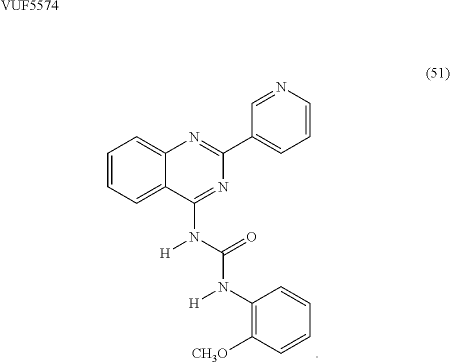

PSB-11 has Chemical Structure (52):

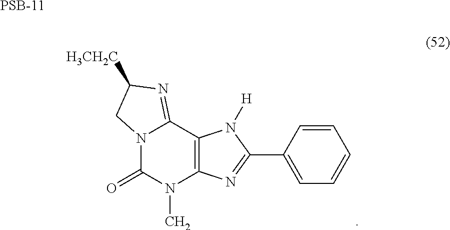

MRS3777 has Chemical Structure (53):

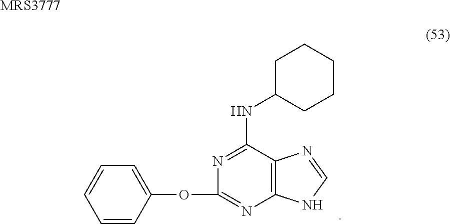 [SMILES: "MRS3777" ; c1ccc(Oc2nc(NC3CCCCC3)c3nc[nH]c3n2)cc1]

MRS1334 has Chemical Structure (54):

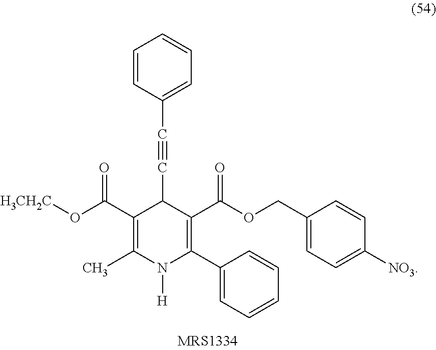

FA385 has Chemical Structure (55):

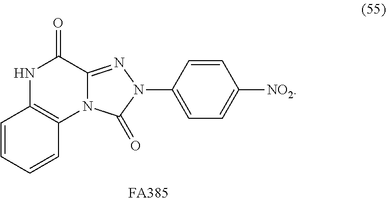

MRE 3008-F20 has Chemical Structure (56):

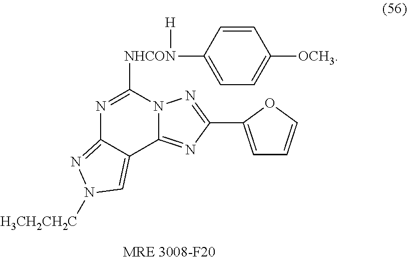

MRS1523 has Chemical Structure (57):

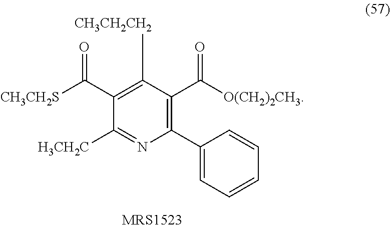

Novartis Compound has Chemical Structure (58):

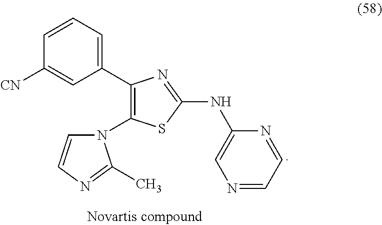

MRS1220 has Chemical Structure (59):

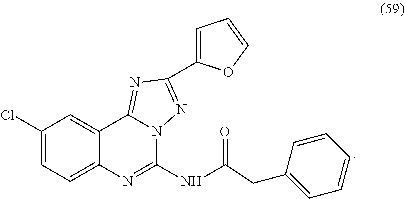 [SMILES: O=C(Cc1ccccc1)Nc1nc2ccc(Cl)cc2c2nc(-c3ccco3)nn12]

In an embodiment, the adenosine receptor antagonist is an A_{2A} adenosine receptor antagonist. The A_{2A} adenosine receptor antagonist may be any suitable pharmaceutically active agent antagonist for the A_{2A} adenosine receptor; see, for example, formula (VII) at column 5, lines 35-67 of U.S. Pat. No. 6,579,868, which is incorporated herein by reference. In an embodiment, the A_{2A} adenosine receptor antagonist is a compound of general formula (XIII):

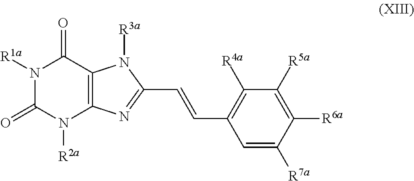

wherein R^{1a} and R^{2a} are the same or different and each represents a C₁₋₄ lower alkyl group or allyl group; R^{3a} represents hydrogen atom or a C₁₋₃ lower alkyl group; and R^{4a}, R^{5a}, R^{6a} and R^{7a} are the same or different and each represents hydrogen atom, a halogen atom, a C₁₋₃ lower alkyl group, a C₁₋₃ lower alkoxy group, nitro group, amino group or hydroxyl group, or isomers, diastereomers, pharmaceutically acceptable salts or solvates of said compound.

In accordance with an embodiment of the invention, the A_{2A} adenosine receptor antagonist is selected from the group consisting of KW6002 (8-[(E)-2-(3,4-dimethoxyphenyl)vinyl]-1,3-diethyl-7-methyl-3,7-dihydro-1H-purine-2,6-dione); CSC; SCH 58261 (7-(2-phenylethyl)-5-amino-2-(2-furyl)-pyrazolo-[4,3-e]-1,2,4-triazolo[1,5-c]pyrimidine); SCH 442416; ZM241,385 (4-(2[7-amino-2-(2-furyl)[1,2,4]triazolo[2,3-a][1,3,5]triazin-5-ylamino]ethyl)-phenol); VER 6947 (2-amino-N-benzyl-6-(furan-2-yl)-9H-purine-9-carboxamide); VER 7835; and Schering compound.

KW6002 has Chemical Structure (18):

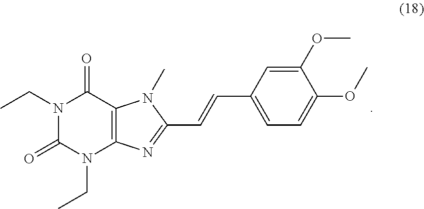 [SMILES: CCn1c(=O)c2c(nc(/C=C/c3ccc(OC)c(OC)c3)n2C)n(CC)c1=O]

CSC has Chemical Structure (19):

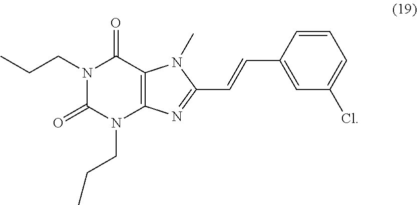 [SMILES: CCCn1c(=O)c2c(nc(/C=C/c3cccc(Cl)c3)n2C)n(CCC)c1=O]

SCH 58261 has Chemical Structure (20):

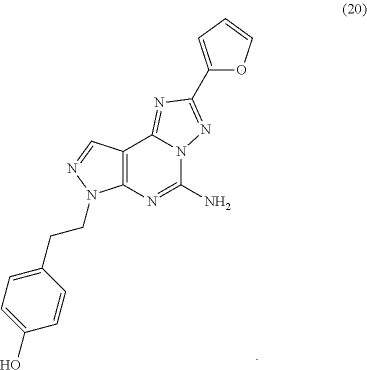 [SMILES: Nc1nc2c(cnn2CCc2ccc(O)cc2)c2nc(-c3ccco3)nn12]

SCH 442416 has Chemical Structure (21):

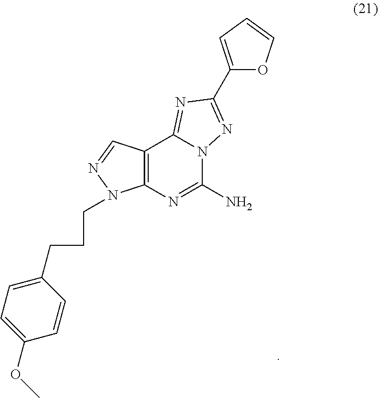 [SMILES: COc1ccc(CCCn2ncc3c2nc(N)n2nc(-c4ccco4)nc32)cc1]

ZM241,385 has Chemical Structure (22):

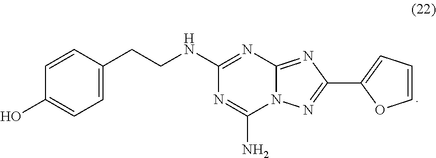 [SMILES: Nc1nc(NCCc2ccc(O)cc2)nc2nc(-c3ccco3)nn12]

VER 6947 has Chemical Structure (23):

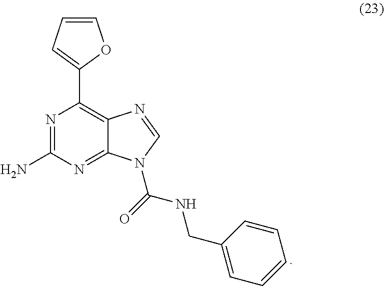 [SMILES: Nc1nc(-c2ccco2)c2ncn(C(=O)NCc3ccccc3)c2n1]

VER 7835 has Chemical Structure (24):

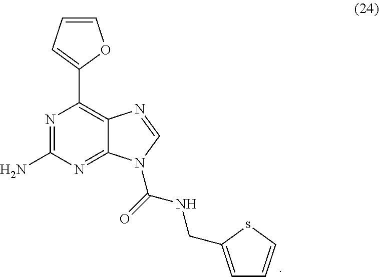

Schering Compound has Chemical Structure (25):

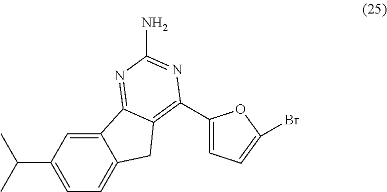 [SMILES: CC(C)c1ccc2c(c1)-c1nc(N)nc(-c3ccc(Br)o3)c1C2]

The A_{2B} adenosine receptor antagonist may be any suitable pharmaceutically active agent antagonist for the A_{2B} adenosine receptor; see, for example, formula (I) at column 5, line 53 to column 7, line 8 of U.S. Pat. No. 6,545,002, which is incorporated herein by reference. In an embodiment, the A_{2B} adenosine receptor antagonist is a compound of general formula (IV):

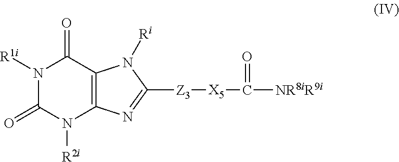

wherein Rⁱ and R¹ⁱ are independently hydrogen, (C₁-C₈)alkyl, (C₂-C₈)alkenyl, (C₂-C₈)alkynyl, (C₁-C₈)alkoxy, (C₃-C₈)cycloalkyl, (C₄-C₁₆)cycloalkylalkyl, heterocycle, (C₆-C₁₀)aryl, (C₇-C₁₈)aralkyl or heteroaryl;

Z₃ is

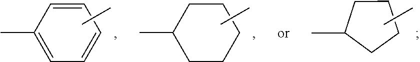

X₅ is (C₁-C₈)alkylene, (C₂-C₈)alkenylene, (C₂-C₈)alkynylene, wherein one of the carbon atoms in the alkylene, alkenylene or alkynylene groups is optionally replaced with a group having the formula —O—, —N(R⁴ⁱ)C(O)—, —OC(O)—, —S—, —S(O)— or —SO₂—,

R²ⁱ is hydrogen, (C₁-C₈)alkyl, (C₂-C₈)alkenyl, (C₂-C₈)alkynyl, (C₁-C₈)alkoxy, (C₃-C₈)cycloalkyl, (C₄-C₁₆)cycloalkylalkyl, (C₆-C₁₀)aryl, (C₇-C₁₈)aralkyl, heterocycle or heteroaryl; wherein R²ⁱ other than H, is optionally substituted with one or more substituents selected from the group consisting of —OH, —SH, —NH₂, —NHR⁷ⁱ, —CN, —COOH and —SO₃H, wherein R⁴ⁱ and R⁷ⁱ are independently hydrogen, (C₁-C₈)alkyl, (C₂-C₈)alkenyl, (C₃-C₈)cycloalkyl, (C₆-C₁₀)aryl, (C₇-C₁₈)aralkyl or halo(C₁-C₆)alkyl; and

wherein R⁸ⁱ is hydrogen, (C₃-C₈)cycloalkyl, (C₄-C₁₆)cycloalkylalkyl, (C₇-C₁₈)aralkyl, heterocycle or heteroaryl, each, other than H, optionally substituted with one or more substituents, wherein the substituents independently are oxo, (C₁-C₈)alkyl, halo(C₁-C₆)alkyl, (C₂-C₈)alkenyl, (C₆-C₁₀)aryl, (C₇-C₁₈)aralkyl, heteroaryl, halo, —OR¹⁵ⁱ, —CN, —NO₂, —CO₂R¹⁵ⁱ, —OC(O)R¹⁶ⁱ, —NR¹³ⁱR¹⁴ⁱ, —N(R²³ⁱ)C(O)R²⁴ⁱ, —C(O)NR¹⁷ⁱR¹⁸ⁱ, —SR¹⁹ⁱ, —SO₂R²⁰ⁱ or —SO₃H; or

R⁸ⁱ is (C₆-C₁₀)aryl, optionally substituted with one or more substituents independently selected from the group consisting of (C₁-C₈)alkyl, halo(C₁-C₆)alkyl, (C₂-C₈)alkenyl, (C₇-C₁₈)aralkyl, heteroaryl, —OR¹⁵ⁱ, —CN, —NO₂, —CO₂R¹⁵ⁱ, —OC(O)R¹⁶ⁱ, —C(O)R¹⁶ⁱ, —NR¹³ⁱR¹⁴ⁱ, —N(R²³ⁱ)C(O)R²⁴ⁱ, —C(O)NR¹⁷ⁱR¹⁸ⁱ, —SR¹⁹ⁱ, —SO₂R²⁰ⁱ and —SO₃H; and

wherein R⁹ⁱ is (C₃-C₈)cycloalkyl, (C₄-C₁₆)cycloalkylalkyl, heterocycle or heteroaryl, each optionally substituted with one or more substituents, wherein the substituents independently are oxo, (C₁-C₈)alkyl, halo(C₁-C₆)alkyl, (C₂-C₈)alkenyl, (C₆-C₁₀)aryl, (C₇-C₁₈)aralkyl, heteroaryl, —OR¹⁵ⁱ, halo, —CN, —NO₂, CO₂R¹⁵ⁱ, —OC(O)R¹⁶ⁱ, —C(O)R¹⁶ⁱ, —NR¹³ⁱR¹⁴ⁱ, —N(R²³ⁱ)C(O)R²⁴ⁱ, —C(O)NR¹⁷ⁱR¹⁸ⁱ, —SR¹⁹ⁱ, —SO₂R²⁰ⁱ or —SO₃H; or

R⁹ⁱ is (C₆-C₁₀)aryl, optionally substituted with one or more substituents independently selected from the group consisting of (C₁-C₈)alkyl, halo(C₁-C₆)alkyl, (C₂-C₈)alkenyl, (C₇-C₁₈)aralkyl, heteroaryl, —OR¹⁵ⁱ, —CN, —NO₂, O₂R¹⁵ⁱ, —OC(O)R¹⁶ⁱ, —C(O)R¹⁶ⁱ, —NR¹³ⁱR¹⁴ⁱ, —N(R²³ⁱ)C(O)R²⁴ⁱ, —C(O)NR¹⁷ⁱR¹⁸ⁱ, —SR¹⁹ⁱ, and —SO₂R²⁰ⁱ, and

wherein R¹³ⁱ, R¹⁴ⁱ, R¹⁵ⁱ, R¹⁶ⁱ, R¹⁷ⁱ, R¹⁸ⁱ, R¹⁹ⁱ, R²⁰ⁱ, R²³ⁱ and R²⁴ⁱ are independently hydrogen, (C₁₋₈)alkyl, (C₂-C₈)alkenyl, (C₃-C₈)cycloalkyl, (C₆-C₈)aryl, (C₇-C₁₈)aralkyl or halo(C₁-C₆)alkyl; provided that when Rⁱ and R⁸ⁱ are both H, R¹ⁱ and R²ⁱ are both alkyl; or isomers, diastereomers, pharmaceutically acceptable salts or solvates of said compound.

In accordance with an embodiment of the invention, the A_{2B} adenosine receptor antagonist is selected from the group consisting of MRS1754 (N-(4-cyanophenyl)-2-[4-(2,6-dioxo-1,3-dipropyl-7H-purin-8-yl)phenoxy]acetamide); MRE 2029-F20 ([N-benzo[1,3]dioxol-5-yl-2-[5-(2,6-dioxo-1,3-dipropyl-2,3,6,7-tetrahydro-1H-purin-8-yl)-1-methyl-1H-pyrazol-3-yloxy]-acetamide]); OSIP-339391 (N-[2-[[2-phenyl-6-[4-(3-phenylpropyl)piperazine-1-carbonyl]-7H-pyrrolo[2,3-d]pyrimidin-4-yl]amino]ethyl]acetamide); and Eisai compound.

MRS1754 has Chemical Structure (26):

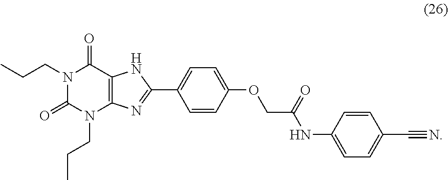 [SMILES: CCCn1c(=O)c2[nH]c(-c3ccc(OCC(=O)Nc4ccc(C#N)cc4)cc3)nc2n(CCC)c1=O]

MRE 2029-F20 has Chemical Structure (27):

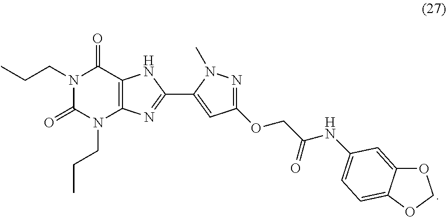 [SMILES: CCCn1c(=O)c2[nH]c(-c3cc(OCC(=O)Nc4ccc5c(c4)OCO5)nn3C)nc2n(CCC)c1=O]

OSIP-339391 has Chemical Structure (28):

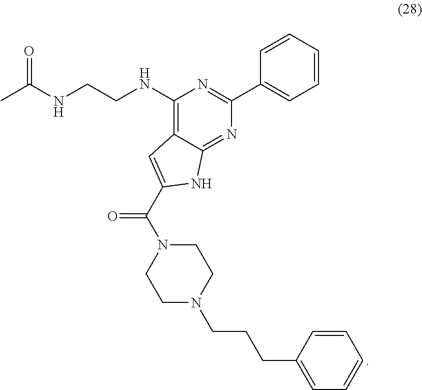 [SMILES: CC(=O)NCCNc1nc(-c2ccccc2)nc2[nH]c(C(=O)N3CCN(CCCc4ccccc4)CC3)cc12]

Eisai Compound has Chemical Structure (29):

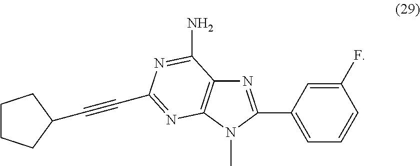 [SMILES: Cn1c(-c2cccc(F)c2)nc2c(N)nc(C#CC3CCCC3)nc21]

Other suitable adenosine receptor agonists and adenosine receptor antagonists are known in the art. Suitable adenosine receptor agonists and adenosine receptor antagonists are disclosed, for example, in U.S. Pat. Nos. 5,688,774; 5,773,423; 6,066,642; 6,329,349; 6,545,002; 6,579,868; 6,586,413; 7,064,112; 7,087,589 (Methanocarba cycloakyl nucleoside analogues are characterized by the combination of adenine or uracil, or their derivatives, with a ring-constrained cycloalkyl group); U.S. Pat. Nos. 7,199,127; 6,376,521; 7,655,637; 6,326,390; 6,605,601; 7,022,686; 5,668,139; 7,576,069; 7,737,127; U.S. Patent Application Publications 2007/0232626; 2009/0012035; 2005/0059683; 2009/0221649; 2005/0119289; 2009/0099212; and World Intellectual Property Organization Publication Nos. 2000/40251; 2009/123881; and 2010/014921, each of which is incorporated herein by reference.

The adenosine receptor agonists and adenosine receptor antagonists described herein may be made by any suitable method known in the art. Exemplary methods for making adenosine receptor agonists and adenosine receptor antagonists are disclosed in U.S. Pat. Nos. 5,688,774; 5,773,423; 6,066,642; 6,329,349; 6,545,002; 6,579,868; 6,586,413; 7,064,112; 7,087,589 (Methanocarba cycloakyl nucleoside analogues are characterized by the combination of adenine or uracil, or their derivatives, with a ring-constrained cycloalkyl group); U.S. Pat. Nos. 7,199,127; 6,376,521; 7,655,637; 6,326,390; 6,605,601; 7,022,686; 5,668,139; 7,576,069; 7,737,127; U.S. Patent Application Publications 2007/0232626; 2009/0012035; 2005/0059683; 2009/0221649; 2005/0119289; 2009/0099212; and World Intellectual Property Organization Publication Nos. 2000/40251; 2009/123881; and 2010/014921, each of which is incorporated herein by reference.

The term “aryl” refers to aromatic moieties, e.g., C₆-C₁₄ aromatic groups, such as phenyl, naphthyl, anthracenyl, and biphenyl. The terms “heterocycle” and “heterocyclic” refer to 3-7 membered rings which can be saturated or unsaturated or heteroaromatic, comprising carbon and one or more heteroatoms such as O, N, and S, and optionally hydrogen; optionally in combination with one or more aromatic rings. Examples of heterocycle groups include pyridyl, piperidinyl, piperazinyl, pyrazinyl, pyrolyl, pyranyl, tetrahydropyranyl, tetrahydrothiopyranyl, pyrrolidinyl, furanyl, tetrahydrofuranyl, thienyl, furyl, thiophenyl, tetrahydrothiophenyl, purinyl, pyrimidinyl, thiazolyl, thiazolidinyl, thiazolinyl, oxazolyl, tetrazolyl, tetrazinyl, benzoxazolyl, morpholinyl, thiomorpholinyl, quinolinyl, and isoquinolinyl. Examples of heteroaryl alkyl include heteroaryl methyl such as 2- or 3-methyl substituted groups, e.g., thienylmethyl, pyridylmethyl, and furylmethyl.

The alkyl, alkoxy, and alkylamino groups can be linear or branched. When an aryl group is substituted with a substituent, e.g., halo, amino, alkyl, hydroxyl, alkoxy, and others, the aromatic ring hydrogen is replaced with the substituent and this can take place in any of the available hydrogens, e.g., 2, 3, 4, 5, and/or 6-position wherein the 1-position is the point of attachment of the aryl group in the compound of the present invention.

The term “halo” refers to fluorine, chlorine, bromine, and iodine.

The phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977). For example, they can be a salt of an alkali metal (e.g., sodium or potassium), alkaline earth metal (e.g., calcium), or ammonium of salt.

Examples of pharmaceutically acceptable salts for use in the present inventive pharmaceutical composition include those derived from mineral acids, such as hydrochloric, hydrobromic, phosphoric, metaphosphoric, nitric and sulphuric acids, and organic acids, such as tartaric, acetic, citric, malic, lactic, fumaric, benzoic, glycolic, gluconic, succinic, maleic and arylsulfonic, for example, benzenesulfonic and p-toluenesulfonic, acids.

The method comprises administering the pharmaceutically active agent(s) described herein to a mammal in an amount effective to treat or prevent a disorder. In a preferred embodiment, the disorder is a vascular and/or joint capsule calcification disorder. In a particularly preferred embodiment, the disorder is a medial vascular and/or joint capsule calcification disorder. Exemplary vascular and/or joint capsule calcification disorders that may be treated or prevented by the inventive methods include, but are not limited to, calciphylaxis, atherosclerosis, Monckeberg's medial sclerosis, ACDC, Ehlers Danlos syndrome, Marfan syndrome, Loewe Dietz syndrome, fibromuscular dysplasia, Kawasaki syndrome, pseudoxanthoma elasticum, and premature placental calcification. In an embodiment, the disorder does not include atherosclerosis.

An “effective amount” or “an amount effective to treat or prevent” refers to a dose that is adequate to treat or prevent vascular calcification, joint capsule calcification, and/or any of the disorders described herein in a mammal. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the pharmaceutically active agent selected, method of administration, timing and frequency of administration, the existence, nature, and extent of any adverse side-effects that might accompany the administration of a particular pharmaceutically active agent, and the desired physiological effect. It will be appreciated by one of skill in the art that various diseases or disorders could require prolonged treatment involving multiple administrations, perhaps using the pharmaceutically active agent(s) described herein in each or various rounds of administration. By way of example and not intending to limit the invention, the dose of the pharmaceutically active agent(s) described herein for methods of preventing vascular and/or joint calcification disorders can be about 0.001 to about 1 mg/kg body weight of the subject being treated per day, for example, about 0.001 mg, 0.002 mg, 0.005 mg, 0.010 mg, 0.015 mg, 0.020 mg, 0.025 mg, 0.050 mg, 0.075 mg, 0.1 mg, 0.15 mg, 0.2 mg, 0.25 mg, 0.5 mg, 0.75 mg, or 1 mg/kg body weight per day. The dose of the pharmaceutically active agent(s) described herein for methods of treating vascular and/or joint calcification disorders can be about 1 to about 1000 mg/kg body weight of the subject being treated per day, for example, about 1 mg, 2 mg, 5 mg, 10 mg, 15 mg, 0.020 mg, 25 mg, 50 mg, 75 mg, 100 mg, 150 mg, 200 mg, 250 mg, 500 mg, 750 mg, or 1000 mg/kg body weight per day.

For purposes of the invention, the amount or dose of the adenosine receptor agonists or adenosine receptor antagonist administered should be sufficient to effect a therapeutic or prophylactic response in the subject or animal over a reasonable time frame. For example, the dose of the pharmaceutically active agent(s) should be sufficient to decrease TNAP activity, increase inorganic phosphate levels, increase extracellular adenosine levels, treat or prevent vascular calcification, joint capsule calcification, and/or disorder in a period of from about 2 hours or longer, e.g., about 12 to about 24 or more hours, from the time of administration. In certain embodiments, the time period could be even longer. The dose will be determined by the efficacy of the particular pharmaceutically active agent(s) and the condition of the animal (e.g., human), as well as the body weight of the animal (e.g., human) to be treated.

The pharmaceutically active agent(s) described herein may be administered in any suitable manner. For example, the pharmaceutically active agent(s) may be administered orally, by inhalation, parenterally, subcutaneously, intravenously, intraarterially, intramuscularly, interperitoneally, intrathecally, rectally, topically, or vaginally.

The pharmaceutically active agent(s) described herein may be administered as a pharmaceutical composition comprising the pharmaceutically active agent(s) and a pharmaceutically acceptable carrier. The pharmaceutically acceptable carrier can be any of those conventionally used and is limited only by chemico-physical considerations, such as solubility and lack of reactivity with the compound, and by the route of administration. It will be appreciated by one of skill in the art that, in addition to the following described pharmaceutical compositions; the compounds of the present invention can be formulated as inclusion complexes, such as cyclodextrin inclusion complexes, or liposomes.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can include (a) liquid solutions, such as an effective amount of the active agent dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The pharmaceutically active agent(s) described herein, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The pharmaceutically active agent(s) described herein may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Additionally, the pharmaceutically active agent(s) described herein may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of vascular and/or joint capsule calcification and/or any of the disorders described herein in a mammal. Furthermore, the treatment or prevention provided by the inventive method can include treatment or prevention of one or more conditions or symptoms of the vascular and/or joint capsule calcification or disorder being treated or prevented. Also, for purposes herein, “prevention” can encompass delaying the onset of the disorder, or a symptom or condition thereof.

Without being bound to a particular theory or mechanism, it is believed that CD73 deficiency results in reduced concentrations of extracellular adenosine that, in turn, increase tissue-nonspecific alkaline phosphatase (TNAP) activity. Without being bound to a particular theory or mechanism, it is believed that increased TNAP activity reduces pyrophosphatase (PPi) which, in turn, leads to calcification.

Another embodiment of the invention provides an adenosine receptor agonist or an adenosine receptor antagonist, either alone or in combination, for use in treating or preventing a disorder selected from the group consisting of atherosclerosis, calciphylaxis, Monckeberg's medial sclerosis, ACDC, Ehlers Danlos syndrome, Marfan syndrome, Loewe Dietz syndrome, fibromuscular dysplasia, Kawasaki syndrome, pseudoxanthoma elasticum, or premature placental calcification. In an embodiment, the disorder does not include atherosclerosis. In this regard, the adenosine receptor agonists and an adenosine receptor antagonists described herein are useful for treating disorders characterized by vascular and/or joint calcification, as described herein. All other aspects of the adenosine receptor agonist and adenosine receptor antagonist are as described herein with respect to the other aspects of the invention.

Still another embodiment of the invention provides an adenosine receptor agonist or an adenosine receptor antagonist, either alone or in combination, for use in treating vascular or joint capsule calcification. In an embodiment, the vascular calcification is medial vascular calcification. In this regard, the an adenosine receptor agonists and adenosine receptor antagonists described herein are useful for treating vascular and/or joint calcification, as described herein. All other aspects of the adenosine receptor agonists and adenosine receptor antagonists are as described herein with respect to the other aspects of the invention.

Still another embodiment of the invention provides the use of an adenosine receptor agonist or an adenosine receptor antagonist, either alone or in combination, in the manufacture of a medicament for treating or preventing medial vascular or joint capsule calcification.

An embodiment of the invention provides a method of determining reduced activity or expression level of CD73 protein comprising: (a) determining the activity or expression level of CD73 protein of a biological sample from a mammal, (b) comparing the activity or expression level of the CD73 protein in the mammal with a negative control; and (c) correlating a decrease in activity or expression of the CD73 protein in the mammal as compared to the negative control with a vascular or joint capsule calcification disorder. In an embodiment, the method does not comprise correlating a decrease in activity or expression of the CD73 protein in the mammal as compared to the negative control with a vascular or joint capsule calcification disorder.

The activity or expression level of the CD73 protein of a biological sample from a mammal can be determined by any suitable method. Typically, the activity or expression level of the protein is determined by assaying the activity or expression level of the protein in a biological sample obtained from the mammal. The biological sample can be any suitable sample, such as a sample of body fluid (e.g., blood, blood plasma, blood serum, serous fluid, lymph fluid, saliva, urine, etc) or tissue (e.g., skin tissue). Preferably, the biological sample is CD34+ hematopoietic stem cells. The biological sample also can be any isolated or fractioned component of the foregoing (e.g., isolated DNA, RNA, or protein from a sample of body fluid or tissue).

The activity level of a CD73 protein can be determined by assaying or measuring the affinity of the protein for its substrate, the kinetics of the reaction of the CD73 protein with its substrate, or the rate or degree of catalysis of the substrate. For example, the activity level of a CD73 protein can be determined by assaying or measuring the ability (i.e., catalytic efficiency) of the protein to convert adenosine monophosphate (AMP) to adenosine and/or inorganic phosphate. The enzyme activity of a CD73 protein also can be determined indirectly by detecting abnormal AMP, adenosine, and/or inorganic phsophate levels in a tissue that expresses the CD73 protein. For example, deficient CD73 activity will result in increased AMP levels and/or decreased inorganic phosphate and/or adenosine levels in tissues that normally express CD73. Specific protocols for suitable assays are known in the art.

The expression level of a CD73 protein can be determined on the basis of mRNA or protein quantification. Specific protocols for quantifying mRNA and protein levels are known in the art (e.g., microarray analysis and Western blot). Probes and antibodies useful for such procedures can be generated from the sequences provided herein using routine techniques, some of which are discussed in connection with other aspects of the invention.

The negative (i.e., normal) control can be any suitable control or standard that reflects the activity or expression level of the CD73 protein of interest in a mammal that contains a non-mutant NT5E gene. Typically, the negative control will be the activity or expression level of the CD73 protein in an appropriate mammal with a non-mutant CD73, such as a mammal that does not have vascular and/or joint capsule calcification, any of the disorders described herein, or any other condition associated with a mutation in the NT5E gene. The control also can be provided by a compilation of activity or expression levels of the CD73 protein from a pool of such individuals (e.g., a standardized activity or expression profile of the CD73 protein of interest).

While any decrease in the activity or expression level of the CD73 protein relative to the negative control can be indicative of a vascular and/or joint calcification disorder, it is believed, without wishing to be bound by any particular theory or mechanism, that the likelihood that a vascular and/or joint calcification disorder is present increases with greater deviance from the negative control. Thus, while a positive result can be indicated by a decrease in CD73 activity or expression relative to the negative control of any amount, preferably a positive result is indicated by a decrease of at least about 10% or more (e.g., at least about 15% or more, 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or even about 100%).

An embodiment of the invention provides a method of detecting or diagnosing a vascular or joint capsule calcification disorder comprising (a) obtaining a nucleic acid sample from a mammal; (b) obtaining a 5′ nucleotidase, ecto (NT5E) coding sequence from the nucleic acid sample; (c) comparing the NT5E coding sequence of the nucleic acid in the sample to a NT5E coding sequence of a negative control and obtaining a difference, wherein the difference is due to a mutation in the NT5E coding sequence of the nucleic acid in the sample; and (d) correlating the difference in the NT5E coding sequence of the nucleic acid in the sample as compared to the NT5E coding sequence of the negative control with a vascular or joint capsule calcification disorder. In an embodiment, the method does not comprise correlating the difference in the NT5E coding sequence of the nucleic acid in the sample as compared to the NT5E coding sequence of the negative control with a vascular or joint capsule calcification disorder.

A mutation in NT5E or its gene product can be detected, for instance, by obtaining a sample from the mammal, which can be any sample as previously described that contains genetic material, and isolating and sequencing the NT5E gene or gene product. The sequence of the NT5E gene or gene product can then be compared to the sequence of a known normal (non-mutant) NT5E gene or gene product, as appropriate. Normal, non-mutant NT5E genes include any naturally occurring NT5E gene that produces a fully-functional gene product in vivo at an expression level that is equivalent to the expression level in a normal control. A normal control is as previously defined herein.

By way of example, a normal non-mutant human NT5E gene sequence is referenced by GenBank Accession No. NM_{—}002526. The sequence of the protein gene product of thenormal non-mutant NT5E gene is referenced by GenBank Accession No. NP_{—}002517 (SEQ ID NO: 2). SEQ ID NO: 1 sets forth the mRNA sequence of the NT5E gene without the untranslated regions. Nucleic acid probes suitable for isolating NT5E or its mRNA transcripts can be designed from the nucleic acid sequences provided herein using routine techniques. Similarly, antibodies and antibody fragments suitable for isolating CD73 proteins, especially CD73, are commercially available or known in the art, or can be obtained using the polypeptides encoded by the nucleic acid sequences disclosed herein by routine techniques. Antibodies are further discussed in connection with other aspects of the invention.

The method comprises correlating the difference in the NT5E coding sequence of the nucleic acid in the sample as compared to the NT5E coding sequence of the negative control with a vascular or joint capsule calcification disorder. Any mutation in NT5E or the gene product thereof (e.g., a mutation in an exon of NT5E) can be used as a basis for a positive result in the method of detecting or diagnosing a vascular or joint capsule calcification disorder. Preferably, the gene mutation is a fully or partially inactivating mutation. As used herein, a mutation is fully or partially inactivating if it decreases or eliminates expression of the gene product, as determined by mRNA or protein levels, or causes the gene to express a protein that is partially or completely non-functional in any respect, preferably with respect to the ability to convert AMP to adenosine and inorganic phosphate.

Any difference between the NT5E coding sequence of the nucleic acid in the sample and the negative control can indicate the presence of a mutation in the NT5E coding sequence of the nucleic acid in the sample. Exemplary differences indicative of a mutation include the deletion, substitution, or insertion of any nucleotide in the NT5E coding sequence of the nucleic acid in the sample as compared to the negative control. The mutation may be any type of mutation. Preferably, the mutation is a missense mutation, a nonsense mutation, or an insertion mutation. As used herein, a “nonsense mutation” means a mutation that results in a premature stop codon as compared to the non-mutated NT5E coding sequence and ultimately, a truncated protein gene product. A “missense mutation” means a mutation that results in a different amino acid in the protein gene product as compared to the protein gene product that results from the non-mutated NT5E. An “insertion mutation” means a mutation that inserts one or more nucleotides into the NT5E coding sequence that are not present in the non-mutated NT5E coding sequence.

By way of illustration, several naturally occurring (i.e., non-engineered) mutations have been discovered in NT5E that result in decreased expression levels or reduced activity levels of the CD73 gene product. For example, SEQ ID NO: 9 sets forth the nucleic acid sequence of a naturally occurring mutant NT5E, wherein the cytosine (C) residue normally occurring at nucleotide residue 662 (located in Exon 3) of the non-mutant NT5E (SEQ ID NO: 1) has been substituted with an adenine residue resulting in a premature stop codon (c662C>A; pS221X). SEQ ID NO: 9 comprises SEQ ID NO: 3, which sets forth the nucleic acid sequence of Exon 3 of the naturally occurring mutant NT5E, wherein the cytosine (C) residue normally occurring at nucleotide residue 100 of Exon 3 of non-mutant NT5E (SEQ ID NO: 4)) has been substituted with an adenine residue resulting in the premature stop codon. Similarly, SEQ ID NO: 10 provides the nucleic acid sequence of a naturally occurring mutant NT5E, wherein the guanine (G) residue normally present at residue 1073 (located in Exon 5) of the non-mutant NT5E (SEQ ID NO: 1) has been substituted with an adenine (A) residue (c1073G>A; pC358Y). The gene mutation translates into a substitution of cysteine with tyrosine at amino acid residue 358 of the protein product. SEQ ID NO: 10 comprises SEQ ID NO: 5, which provides the nucleic acid sequence of Exon 5 of the naturally occurring mutant NT5E, wherein the guanine (G) residue normally present at residue 124 of Exon 5 of non-mutant NT5E (SEQ ID NO: 6)) has been substituted with an adenine (A) residue. SEQ ID NO: 11 provides the nucleic acid sequence of a naturally occurring mutant NT5E, wherein an adenine (A) residue not normally present at residue 1609 (located in Exon 9) of the non-mutant NT5E (SEQ ID NO: 1) has been inserted (c1609dupA; V537fsX7), resulting in a premature stop codon. SEQ ID NO: 11 comprises SEQ ID NO: 7, which provides the nucleic acid sequence of Exon 9 of the naturally occurring mutant NT5E, wherein an adenine (A) residue not normally present at residue 48 of Exon 9 of non-mutant NT5E (SEQ ID NO: 8)) has been inserted, resulting in a premature stop codon.

Thus, while the method of detecting or diagnosing a vascular or joint capsule calcification disorder can comprise detecting any mutation in NT5E or its gene product, the method preferably comprises detecting one or more of the forgoing specific mutations or a different mutation in the region of NT5E in which any of the foregoing specific mutations occur. In particular, the method preferably comprises detecting a mutation in one or more of Exons 3, 5, and 9 of NT5E. More particularly, the method can comprise detecting a mutation that comprises a substitution or insertion of a nucleotide residue of non-mutant human NT5E selected from the group consisting of (a) nucleotide residue 100 of Exon 3, (b) nucleotide residue 124 of Exon 5, and (c) nucleotide residue 48 of Exon 9. For instance, the method can comprise detecting a nucleic acid sequence of any one or more of SEQ ID NOs: 3, 5, 7, 9-11, or a nucleic acid sequence that comprises about 85% or greater, (e.g., about 90% or greater, 95% or greater, or even about 99% or greater) sequence identity to any one or more of SEQ ID NOs: 3, 5, 7, and 9-11. Methods for detecting specific mutations, such as the mutations described herein, using the nucleic acid sequences provided in any one or more of SEQ ID NOs: 3, 5, 7, and 9-11 are known in the art and are further discussed in connection with other aspects of the invention.

The vascular or joint capsule calcification disorder may be any of the vascular or joint capsule calcification disorders described herein. Preferably, the vascular or joint capsule calcification disorder is due to CD73 deficiency. In an embodiment of the invention, the vascular calcification is medial vascular calcification.

As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

Still another embodiment of the invention provides a method of treating or preventing vascular or joint capsule calcification in a mammal in need thereof, comprising administering to the mammal a pharmaceutically active agent selected from the group consisting of an A₁ adenosine receptor agonist, an A₃ adenosine receptor agonist, an A_{2A} adenosine receptor agonist, and an A_{2B} receptor agonist in an amount effective to treat or prevent the vascular or joint capsule calcification, wherein the mammal has a NT5E mutation. Accordingly, the pharmaceutically active agents described herein are useful for treating or preventing vascular or joint capsule calcification in a mammal that has a NT5E mutation. All other aspects of the method of identifying an agent that modifies the activity of the polypeptide encoded by a nucleic acid of the invention is as described herein with respect to the other aspects of the invention.

In a related aspect, the invention provides a nucleic acid comprising a mutation in one or more exons of human NT5E selected from the group consisting of Exon 3, Exon 5, and Exon 9. In an embodiment, the nucleic acid is an isolated nucleic acid. More particularly, the mutation can comprise a substitution or insertion of a nucleotide residue of non-mutant human NT5E selected from the group consisting of (a) nucleotide residue 100 of Exon 3, (b) nucleotide residue 124 of Exon 5, and (c) nucleotide residue 48 of Exon 9. Preferably, the nucleic acid comprises a nucleotide sequence selected from the group consisting of SEQ ID NO: 3, SEQ ID NO: 5, SEQ ID NO: 7, SEQ ID NO: 9, SEQ ID NO: 10, SEQ ID NO: 11, or a combination thereof, as well as a polypeptide encoded by the nucleic acid.

Also encompassed by the invention is a nucleic acid comprising a degenerate nucleic acid sequence of any one of SEQ ID NOs: 3, 5, 7, and 9-11 which encode the same protein as encoded by any one of SEQ ID NOs: 3, 5, 7, and 9-11 or a nucleic acid sequence that is otherwise substantially identical to any one of SEQ ID NOs: 3, 5, 7, and 9-11. Substantially identical sequences include any sequence that has a sequence identity to any one of SEQ ID NOs: 3, 5, 7, and 9-11 of 85% or more, preferably 90% or more, or even 95% or more, as determined using available algorithms (e.g., the Basic Local Alignment Search Tool (BLAST) made publicly available through the National Center for Biotechnology Information, Bethesda, Md.).

Similarly, the invention also encompasses a polypeptide encoded by any of the foregoing nucleic acid sequences (e.g., SEQ ID NOs: 3, 5, 7, 9-11, and sequences degenerate to or substantially identical to SEQ ID NOs: 3, 5, 7, and 9-11). In an embodiment, the polypeptide is an isolated polypeptide. In this regard, SEQ ID NO: 12 provides the amino acid sequence of a naturally occurring mutant CD73 protein encoded by SEQ ID NO: 9, wherein the non-mutant CD73 (SEQ ID NO: 2) is truncated at position 221. SEQ ID NO: 13 provides the amino acid sequence of a naturally occurring mutant CD73 protein encoded by SEQ ID NO: 10, wherein the cysteine (C) normally present at residue 358 of non-mutant CD73 (SEQ ID NO: 2) is substituted with tyrosine (Y). Similarly, SEQ ID NO: 14 provides the amino acid sequence of a naturally occurring mutant CD73 protein encoded by SEQ ID NO: 11, wherein the non-mutant CD73 (SEQ ID NO: 2) is truncated at position 543, and the valine (V), ilsoleucine (I), tyrosine (Y), proline (P), alanine (A), and valine (V) normally present at positions 537-542 of non-mutant CD73 (SEQ ID NO: 2), respectively, are substituted with serine (S), asparagine (N), leucine (L), serine (S), serine (S), and serine (S), respectively. The polypeptides of the invention can further comprise one or more conservative amino acid substitutions, provided that such substitutions do not obliterate the function of the polypeptide. Preferably, the number of amino acid substitutions is not greater than 15% of the total number of amino acid residues, more preferably not greater than 10% or even 5% of the amino acid residues. As used herein, the term isolated means removed from its natural environment, synthetically generated, or otherwise engineered.

The nucleic acid can be provided as part of a vector (e.g., a vector comprising the nucleic acid). Any suitable vector can be used. Suitable vectors include nucleic acid vectors, such as naked DNA and plasmids, liposomes, molecular conjugates, and viral vectors, such as retroviral vectors, parvovirus-based vectors (e.g., adenoviral-based vectors and adeno-associated virus (AAV)-based vectors), lentiviral vectors (e.g., Herpes simplex (HSV)-based vectors), and hybrid or chimeric viral vectors, such as an adenoviral backbone with lentiviral components (see, e.g., Zheng et al., Nat. Biotech., 18(2): 176-80 (2000); International Patent Application WO 98/22143; International Patent Application WO 98/46778; and International Patent Application WO 00/17376) and an adenoviral backbone with AAV components (see, e.g., Fisher et al., Hum. Gene Ther., 7: 2079-2087 (1996)). Vectors and vector construction are known in the art (see, e.g., Sambrook et al., Molecular Cloning: A Laboratory Manual, 3^{rd} Edition, Cold Spring Harbor Laboratory Press, New York (2001); and Ausubel et al., Current Protocols in Molecular Biology, Green Publishing Associates and John Wiley & Sons, New York, N.Y. (1994)).

The vector can comprise any suitable promoter and other regulatory sequences (e.g., transcription and translation initiation and termination codons) to control the expression of the nucleic acid sequence. The promoter can be a native or normative promoter operably linked to the nucleic acid described above. The selection of promoters, including various constitutive and regulatable promoters, is within the skill of an ordinary artisan. Examples of regulatable promoters include inducible, repressible, and tissue-specific promoters. Specific examples include viral promoters, such as adenoviral promoters, AAV promoters, and CMV promoters. Additionally, operably linking the nucleic acid described above to a promoter is within the skill in the art.

The nucleic acid or vector can be introduced into a cell, thereby providing a recombinant cell comprising the nucleic acid, optionally in a vector. Any suitable cell (e.g., an isolated cell) can be used. Examples include host cells, such as E. coli (e.g., E. coli Tb-1, TG-2, DH5α, XL-Blue MRF' (Stratagene), SA2821, and Y1090), Bacillus subtilis, Salmonella typhimurium, Serratia marcescens, Pseudomonas (e.g., P. aerugenosa), N. grassa, insect cells (e.g., Sf9, Ea4), yeast (S. cerevisiae) cells, and cells derived from a mammal, including human cell lines. Specific examples of suitable eukaryotic cells include VERO, HeLa, 3T3, Chinese hamster ovary (CHO) cells, W138 BHK, COS-7, and MDCK cells. Methods of introducing vectors into isolated host cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation (see, e.g., Sambrook et al., supra, Davis et al., Basic Methods in Molecular Biology (1986), and Neumann et al., EMBO J. 1: 841 (1982)). Preferably, the recombinant cell expresses the polypeptide encoded by the nucleic acid of the invention.

The recombinant cell can be an isolated cell, a cell of a cell culture, a cell of a tissue, or a cell of a host, such as a mammal or human. In this regard, the invention also provides a recombinant cell that comprises a mutation in one or more exons of NT5E selected from the group consisting of Exon 3, Exon 5, and Exon 9. Preferably, the mutation is an inactivating mutation, such as any one or more of the specific mutations described herein with respect to the method of detecting or diagnosing a vascular or joint capsule calcification disorder. More particularly, the recombinant cell can comprise a nucleic acid having the sequence of any one or more of SEQ ID NOs: 3, 5, 7, and 9-11 optionally in the form of a vector. The recombinant cell can be any suitable cell, as previously described.

The invention further provides an antibody, or an antigen binding portion thereof, that binds to a polypeptide encoded by the nucleic acids described herein. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the polypeptides of the invention. Desirably, the antibody is specific for the polypeptide, such that there is minimal cross-reaction with other peptides or proteins, such as wild-type CD73 protein.

Methods of testing antibodies for the ability to bind to the polypeptides are known in the art and include any antibody-antigen binding assay, such as, for example, radioimmunoassay (RIA), ELISA, Western blot, immunoprecipitation, and competitive inhibition assays (see, e.g., Janeway et al., Immunobiology: The Immune System in Health and Disease, 4^{t1} ed., Current Biology Publications: Garland Publishing, New York, N.Y., 1999)).

Suitable methods of making antibodies are known in the art. For instance, standard hybridoma methods are described in, e.g., Köhler and Milstein, Eur. J. Immunol., 5: 511-519 (1976), Harlow and Lane (eds.), Antibodies: A Laboratory Manual, CSH Press (1988), and C. A. Janeway et al. (eds.), Immunobiology, 5^{th} Ed., Garland Publishing, New York, N.Y. (2001)). Alternatively, other methods, such as EBV-hybridoma methods (Haskard and Archer, J. Immunol. Methods, 74(2): 361-67 (1984), and Roder et al., Methods Enzymol., 121: 140-67 (1986)), and bacteriophage vector expression systems (see, e.g., Huse et al., Science, 246, 1275-81 (1989)) are known in the art. Furthermore, methods of producing antibodies in non-human animals are described in, e.g., U.S. Pat. Nos. 5,545,806, 5,569,825, and 5,714,352.

Phage display also can be used to generate the antibody of the invention. In this regard, phage libraries encoding antigen-binding variable (V) domains of antibodies can be generated using standard molecular biology and recombinant DNA techniques (see, e.g., Sambrook et al., supra). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

Methods for generating humanized antibodies are well known in the art and are described in detail in, for example, Janeway et al., supra, U.S. Pat. Nos. 5,225,539, 5,585,089 and 5,693,761, European Patent No. 0239400 Bl, and United Kingdom Patent No. 2188638. Humanized antibodies can also be generated using the antibody resurfacing technology described in U.S. Pat. No. 5,639,641 and Pedersen et al., J. Mol. Biol., 235: 959-973 (1994).

The invention also provides antigen binding portions of any of the antibodies described herein. The antigen binding portion can be any portion that has at least one antigen binding site, such as Fab, F(ab′)₂, dsFv, scFv, diabodies, and triabodies.

A single chain variable region fragment (scFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7: 697-704 (1994)).

The antibodies can be used, for instance, in a method of determining an NT5E mutation comprising detecting a polypeptide encoded by any of the nucleic acids described herein. For example, such a method can comprise (a) contacting a biological sample with an antibody that binds to a polypeptide encoded by a nucleic acid described herein, but does not bind to a polypeptide encoded by non-mutant NT5E, e.g., a polypeptide comprising SEQ ID NO: 2 (the amino acid sequence of wild-type CD73 protein), (b) detecting the binding of the antibody to the polypeptide, and (c) correlating the binding of the antibody to the polypeptide with a vascular or joint capsule calcification disorder. In an embodiment, the method does not comprise correlating the binding of the antibody to the polypeptide with a vascular or joint capsule calcification disorder. The detection of the binding of the antibody to the polypeptide can be done by any suitable method, such as Western blotting, immunoassays, and immunohistochemistry techniques known in the art. Other aspects of the antibodies and method of detecting or diagnosing a vascular or joint calcification disorder comprising detecting a polypeptide are as described with respect to other aspects of the invention.

The following examples further illustrate the invention but, of course, should not be construed as in any way limiting its scope.

### Example 1

This example demonstrates a method of identification of a mutation in the NT5E gene in individuals suffering from a previously undiagnosed vascular calcification disorder.

### Patients

Members of Families 1 and 3 were admitted to the NIH Undiagnosed Diseases Program and enrolled in clinical protocols approved by the Institutional Review Board of the National Human Genome Research Institute. The genetic studies for Family 2 were approved by Azienda Ospedaliera San Giovanni Battista, Torino, Italy. All subjects gave written, informed consent.

### Case Reports

The proband of Family 1, patient VI-1, was a 54 year-old woman with a 20-year history of intermittent claudication of the calves, thighs, and buttocks and chronic ischemic rest pain of the feet. Her parents were third cousins. Vascular calcifications in her lower extremities were initially diagnosed as tumoral calcinosis and treated weekly with sodium thiosulfate (12.5 mg, IV). A cardiac CT gave a calcium score of zero, with normal coronary arteries, and normal left ventricular systolic function. Upon admission to the NIH Clinical Center, the patient had physical signs of chronic bilateral lower extremity ischemia with dependent rubor, atrophy of the skin, loss of hair and thickened nails, with preserved motor and sensory function. Her ankle-brachial indices (ABIs) were markedly reduced, but serum calcium, phosphate, vitamin D, alkaline phosphatase, creatinine, cholesterol, and other chemistries were normal (Table 1). Contrast-enhanced magnetic resonance angiography revealed extensive iliac, femoropopliteal, and tibial artery occlusion, with extensive collateralization. Plain radiographs of the lower extremities revealed heavy calcification, with areas of arteriomegaly; a chest radiograph revealed no vascular calcifications above the diaphragm. Other radiographs revealed juxta-articular joint capsule calcifications of the fingers, wrists, ankles and feet without obvious joint erosions.

TABLE 1

| | VI-1 | | VI-2 | | VI-3 | | VI-4 | | VI-5 | | Control |
|---|---|---|---|---|---|---|---|---|---|---|---|
| Age/Sex | 54/F | | 53/M | | 51/M | | 49/F | | 44/F | | |
| Calcification | | | | | | | | | | | |
| Coronary arteries | Normal | | Moderate | | Normal | | Normal | | Normal | | Normal |
| Aorta | Normal | | Normal | | Normal | | Normal | | Normal | | Normal |
| Iliac arteries | Calcified | | Mildly | | Tortuous, Mildly | | Minimally | | Calcified | | Normal |
| | Occluded | | Calcified | | Calcified | | Calcified | | Not obstructed | | |
| Femoral arteries | Calcified, | | Calcified, | | Calcified, | | Calcified, | | Calcified, | | Normal |
| | Occluded | | Occluded | | Occluded | | Occluded | | Occluded | | |
| | Popliteal | | Femoropopliteal | | | | Femoropopliteal | | Popliteal | | |
| | Arteriomegaly | | Arteriomegaly | | | | Arteriomegaly | | Arteriomegaly | | |
| Tibial arteries | Calcified, | | Calcified | | Normal | | Calcified, | | Calcified, | | Normal |
| | Occluded | | | | | | Occluded | | Proximal | | |
| | | | | | | | | | Occlusion | | |
| Diabetes Mellitus | No | | No | | No | | No | | No | | No |
| WBC (K/μl) | 3.57 | | 5.20 | | 7.39 | | 4.15 | | 3.56 | | 3.98-10.04 |
| Hgb (g/dL) | 13.5 | | 15.7 | | 13.9 | | 12.4 | | 12.3 | | 11.2-15.7 |
| Calcium (mmol/L) | 2.31 | | 2.30 | | 2.42 | | 2.29 | | 2.29 | | 2.05-2.50 |
| Phosphate (mg/dL) | 3.8 | | 3.2 | | 3.7 | | 3.8 | | 3.4 | | 2.5-4.8 |
| Alk Phos (U/L) | 70 | | 69 | | 62 | | 62 | | 65 | | 37-116 |
| PTH (pg/mL) | 102 | | 59.4 | | 18.6 | | 39.7 | | 24.9 | | 16.0-87.0 |
| Vit D 1.25 (pg/mL) | 72 | | 55 | | 37 | | 53 | | 58 | | 18-78 |
| Creatinine (mg/dL) | 0.70 | | 0.82 | | 0.73 | | 0.40 | | 0.54 | | 0.70-1.30 |
| Cholesterol (mg/dL) | 182 | | 109 | | 143 | | 204 | | 153 | | <200 |
| ABI | R | L | R | L | R | L | R | L | R | L | 1.00-1.29 |
| | 0.43 | 0.38 | 0.75 | 0.53 | 0.67 | 0.71 | 0.27 | 0.34 | 0.69 | 0.75 | |

The patient's sister, VI-4, was a 49 year-old woman with intermittent calf claudication and nocturnal metatarsalgia for ten years. She was receiving weekly sodium thiosulfate (12.5 mg, IV) and daily clopidogrel. Radiographs in 2007 showed vascular and peri-articular calcifications in the shoulders, elbows, hands, hips, and knees. A coronary artery calcium score was zero. At the NIH, the ABI was reduced, and echocardiogram, electrocardiogram, and blood chemistries (Table 1) were normal. The vascular findings of the three remaining siblings (VI-2, VI-3, VI-5) closely resembled those of their sisters. Whole body CT calcium scans demonstrated prominent circumferential vascular calcifications of the femoral, popliteal and proximal tibial arteries, also involving the iliac artery and abdominal aorta in patient VI-5. Notably absent were calcifications in the coronary arteries, except for patient VI-2, who had moderate coronary calcifications (Agatston score (Agatston et al. J. Am. Coll. Cardiol. 15:827-32 (1990)): 145).

All five siblings had disabling intermittent claudication (able to walk 1-6 blocks) and hemodynamically significant lower extremity obstructive peripheral artery disease, with normalized resting ABIs between 0.3 and 0.8 (normal 1.0-1.2). All had extensive femoropopliteal occlusion evident on magnetic resonance arteriography with diffuse, mild aneurysmal remodeling in a pattern of arteriomegaly. CT angiography in three revealed the obstructive lesions to be diffusely and heavily calcified. Four siblings had joint pain and swelling; past evaluations for rheumatoid arthritis and other joint-related autoimmune disorders were negative.

The proband of Family 2, patient II-4, was a 68 year-old Italian female whose mother's surname was present among her father's relatives four generations ago. She reported intense joint pain in her hands, unresponsive to corticosteroids administered from age 14-27 years. Radiographs of the lower limbs revealed calcifications, initially diagnosed as chondrocalcinosis. Serum electrolytes, calcium, and cholesterol were normal. Two sisters, aged 73 and 70 years, exhibited lower extremity pain and vascular calcifications similar to those of the proband.

The proband of Family 3, patient II-1, was a 44 year-old female of Northern European ancestry. At age 19 years, a peri-articular calcification in the right first metatarsal joint was surgically excised. At age 27, partial generalized seizures occurred; a calcified oligodendroglioma was removed from the left temporal lobe at age 29. Dystrophic calcifications were noted in wrist films at age 26 years; at age 41 years, swelling and severe pain in the hands and wrists were diagnosed as pseudogout. At age 42 years, mild paresthesias in the lower legs prompted an evaluation that revealed extensive calcifications of the distal arteries, with sparing of the carotids, aorta and coronaries. Extensive rheumatology evaluations were negative. Concern for impending ischemia to the right leg prompted a femoral popliteal bypass at age 43 years. Serum C-reactive protein, cholesterol, lipids, calcium, phosphate and vitamin D levels were within normal ranges.

None of the 9 affected patients had diabetes mellitus type 2, decreased kidney function, or parents or children similarly affected. Bone morphology appeared normal.

### SNP Array Analysis

Genomic DNA from Family 1 was isolated from peripheral leucocytes and genotyped on an Illumina 1 M Duo genotyping array. Minor (B) allele plots were generated using GenomeStudio. Anomalous regions of homozygosity were identified visually and confirmed by haplotype imputation (ENT program, U. Conn). An estimated LOD score was established using parametric multipoint linkage analyses, as described (Bockenhauer et al. N. Engl. J. Med. 360:1960-70 (2009)).

### Mutation Analysis of CD73

Coding exons and intron-exon junctions of NT5E were amplified using a touchdown PCR containing 50 ng of genomic DNA, 3 μM of sense and antisense oligonucleotides, and 5 μl of HotStart Master Mix (Qiagen) in a final volume of 104 PCR products were sequenced in both directions using the Big Dye terminator kit v1.1 (Applied Biosystems) and an automated capillary sequencer (ABI PRISM 3130×1 Genetic Analyzer, Applied Biosystems). Electrophoretogram-derived sequences were compared to reference sequences for NT5E (Ensembl gene number ENSG00000135318) using Sequencher software 4.8. Screening of 200 Caucasian control DNAs (Coriell Repository, Camden, N.J.) was perfoiined using the 5′ nuclease allelic discrimination (TaqMan) assay, as described (Livak Genet. Anal. 14:143-9 (1999)).

### Expression Studies

RNA was isolated from cultured fibroblasts using the RNAeasy® kit (Qiagen) and 1 μg was used to generate cDNA with the Superscript II reverse transcriptase kit (Invitrogen). Expression of NT5E was measured by quantitative real-time polymerase chain reaction (qPCR) employing SYBR Green technology on a Chroma4 Real Time PCR system (Bio-Rad). Expression levels were calculated using the 2^{-ΔCt} method, where the cycling threshold (Ct) of the candidate gene was compared to the Ct of 18s rRNA and expressed as a power of two (2^{(Ct-CD73-Ct-18s)). Primers: NT}5E, FP: 5′-GGGCGGAAGGTTCCTGTAG-3′ (SEQ ID NO: 15); RP: 5′-GAGGAGCCATCCAGATAGACA-3′(SEQ ID NO: 16); 18s rRNA, FP: 5′-GTAACCCGTTGAACCCCATT-3′ (SEQ ID NO: 17); RP: 5′-CCATCCAATCGGTAGTAGCG-3′ (SEQ ID NO:18).

### Results of the SNP Array, Mutation Analyses, and Expression Studies

The consanguineous pedigree of Family 1, with disease confined to one generation, suggested autosomal recessive inheritance. Therefore, regions of homozygosity/identity by descent present in all five affected siblings but absent from their parents were investigated. Only one such region, with an estimated LOD score of 4.51, was found across the entire genome, on chromosome 6q14, encompassing 22.4 MB (86,157,551 to 108,573,717), defined by 7977 genotyped SNPs, and containing 92 genes.

Three reasonable candidate genes were selected, but direct sequencing identified a homozygous nonsense mutation (c662C>A; pS221X) in exon 3 of the NT5E gene of all five siblings of Family 1, and the same nonsense mutation in the heterozygous state in both parents. Quantitative PCR analysis documented decreased NT5E mRNA expression in the fibroblasts of patients VI-1 and VI-4 (NT5E normalized to 18s was less than approximately 0.1 for both of patients VI-4 and VI-1, approximately 0.8 for Control 1 (Ct-1), and approximately 1.0 for Control 2 (Ct-2)). Affected members of Family 2 were homozygous for a missense mutation, c1073G>A (pC358Y), in exon 5 of NT5E; the mother was heterozygous for this variant of an amino acid that is conserved across 16 species. The affected member of Family 3 was compound heterozygous for the c662C>A nonsense mutation found in Family 1 and a c1609dupA (V537fsX7) mutation leading to a premature stop codon in exon 9 of NT5E. None of these mutations was present in 400 ethnically matched (Caucasian) control alleles.

This example demonstrated that individuals suffering from a previously undiagnosed, inherited vascular calcification disorder have a mutation in the NT5E gene that leads to a decrease of NT5E mRNA expression.

### Example 2

This example demonstrates that fibroblasts having a NT5E mutation have reduced CD73 protein expression.

NT5E encodes CD73, a membrane-bound ecto-5-prime-nucleotidase (5-prime-ribonucleotide phosphohydrolase; EC 3.1.3.5) involved in extracellular ATP metabolism. The enzyme preferentially binds AMP and converts it to adenosine and inorganic phosphate. CD73 protein expression in the fibroblasts of patients VI-1 and VI-4 of Family 1 of Example 1 were analyzed by Western blot.

### Fibroblast Cell Cultures

Fibroblast cultures were prepared from a 4 mm punch skin biopsy and grown in DMEM containing 10% FBS and 1% penicillin/streptomycin, as described (Normand et al. In Vitro Cell Dev. Biol. Anim. 31:447-55 (1995)). Cells were fed every other day and split 1:2 upon confluency.

### Western Blot

Cells were grown to confluency, trypsinized, pelleted, and lysed by addition of 50 mM Tris-HCl pH7.4, 150 mM NaCl, 2 mM EDTA, 1% NP-40 and 0.1% SDS (RIPA buffer) supplemented with 0.5% Triton-x 100 and 1× Complete Mini Protease Inhibitor Cocktail (Roche). After 10 min on ice, the lysate was vortexed at 4° C. for 5 min and centrifuged at 15,000×g and supernatant protein quantified using the bicinchoninnic acid assay (Pierce). Thirty μg of protein was mixed with SDS protein gel loading solution (Quality Biologicals), loaded on a 4-20% polyacrylamide gel (Bio-Rad), and electrophoresed at 120V for 1.5 h. After protein transfer, antibodies against CD73 (Abeam) and actin (Sigma-Aldrich) were used at dilutions of 1:1000 and 1:50,000, respectively.

Protein analysis using Western blotting of fibroblast extracts of patients VI-1 and VI-4 of Family 1 demonstrated absent CD73 protein compared to normal control patients.

This example demonstrated that the NT5E mutations described in Example 1 result in decreased CD73 protein expression.

### Example 3

This example demonstrates that fibroblasts having a NT5E mutation have decreased CD73 enzyme activity.

The CD73 enzyme activity in the fibroblasts of patients VI-1 and VI-4 of Family 1 of Example 1 were analyzed by CD73 enzyme assay.

Fibroblasts were washed with a solution of 2 mM MgCl₂, 120 mM NaCl, 5 mM KCl, 10 mM glucose and 20 mM HEPES. Incubation buffer, consisting of wash buffer supplemented with 2 mM AMP, was added and cells were incubated at 37° C. for 10 min (Deaglio et al. J. Exp. Med. 204:1257-65 (2007)). The supernatant was removed and inorganic phosphate measured with the Sensolyte® MG Phosphate Assay Kit (Anaspec), according to the manufacturer's instructions. Inorganic phosphate was normalized to μg protein.

An assay of CD73 in fibroblasts of these patients revealed virtually absent enzymatic activity. The results are shown in Table 2.

TABLE 2

| | | Approximate Inorganic |
|---|---|---|
| | Patient | Phosphate (μg/μg protein) |
| Affected | VI-4 | 0 |
| | VI-1 | 500 |
| Controls | C-1 | 4800 |
| | C-2 | 4500 |
| | C-3 | 4000 |

This example demonstrated that the NT5E mutations described in Example 1 result in decreased CD73 enzyme activity.

### Example 4

This example demonstrates that normal AMP-dependent inorganic phosphate production can be restored in a fibroblast having the NT5E mutation by genetic rescue with a vector encoding CD73.

The fibroblasts of patient VI-4 of Family 1 of Example 1 were transduced with a CD73-encoding lentiviral vector.

The pCMV-Sport6 plasmid containing human CD73 cDNA was purchased from Open Biosystems. Since the pCMV-Sport6 vector contains Gateway® Cloning (Invitrogen) recombination sites, this cloning strategy was used to insert normal CD73 cDNA into the pLenti6.3/V5-DEST vector; lentivirus was generated using the ViraPower™ HiPerform™ Lentiviral Gateway® Expression Kit (Invitrogen). For transduction, viral particles were added to cells in growth medium containing 6 μg/mL polybrene (Sigma). To select for cells transduced with virus, blasticidin (Invitrogen, 10 μg/mL) was added to growth media 4 days post transduction. The CD73 enzyme activity was measured by CD73 enzyme assay as described in Example 3.

Genetic rescue with a CD73-encoding lentiviral vector re-established normal AMP-dependent inorganic phosphate production. The results are shown in Table 3.

TABLE 3

| | | Approximate Inorganic |
|---|---|---|
| | Vector | Phosphate (μg/μg protein) |
| Control | GFP | 5000 |
| | CD73 | 13,000 |
| Affected Patient VI-4 | GFP | 0 |
| | CD73 | 10,000 |

This example demonstrated that the transduction of the fibroblasts of a patient having an NT5E mutation with a vector encoding CD73 re-established normal AMP-dependent inorganic phosphate production.

### Example 5

This example demonstrates that cells transfected with mutated NT5E have decreased CD73 enzyme activity as compared to cells transfected with wild-type NT5E.

The enzymatic activities of normal and mutant CD73 constructs were tested in CD73-deficient HEK293 cells.

The pCMV-Sport6 plasmid containing human CD73 cDNA was purchased from Open Biosystems. Mutations were introduced using the QuikChange® XL Site-Directed Mutagenesis Kit (Stratagene) with the following primers: Family 1: FP: 5′-(SEQ ID NO:19)-3′; RP: 5′-(SEQ ID NO: 20)-3′; Family 2: FP: 5′-(SEQ ID NO: 21)-3′; RP: 5′-(SEQ ID NO: 22)-3′; Family 3: FP: 5′-(SEQ ID NO: 23)-3′; RP: 5′-(SEQ ID NO: 24)-3′. Construct sequences were confirmed by sequencing in both directions. Since the pCMV-Sport6 vector contains Gateway® Cloning (Invitrogen) recombination sites, this cloning strategy was used to insert normal and mutated CD73 cDNA into the pLenti6.3/V5-DEST vector; lentivirus was generated using the ViraPower™ HiPerform™ Lentiviral Gateway® Expression Kit (Invitrogen). The CD73 enzyme activity was measured by CD73 enzyme assay as described in Example 3.

HEK293 cells were cultured in DMEM supplemented with 10% FBS and 1% penicillin/streptomycin. One μg of CMV-Sport6 plasmid containing GFP, wild-type NT5E cDNA, or mutated NT5E cDNA (c662C>A; c1073G>A, c1609dupA; c662C>A & c1609dupA), was transfected into HEK293 cells using FuGENE®6 (Roche) reagent according to the manufacturer's instructions. Three days post transfection, cells were analyzed for CD73 activity as described above.

Transfection with normal NT5E cDNA produced abundant CD73 activity, while transfection with the c662C>A, c1073G>A, and c1609dupA NT5E yielded negligible production of AMP-dependent inorganic phosphate. The results are shown in Table 4.

TABLE 4

| | Approximate Inorganic |
|---|---|
| Vector | Phosphate (μg/μg protein) |
| Control GFP Vector | 200 |
| wild-type NT5E | 4000 |
| c662C>A | 200 |
| c1073G>A | 200 |
| c1609dupA | 250 |
| c662C>A & c1609dupA | 225 |

This example demonstrated that cells transfected with mutated NT5E demonstrate decreased AMP-dependent inorganic phosphate production as compared to cells transfected with wild-type NT5E.

### Example 6

This example demonstrates that adenosine treatment of fibroblasts having the NT5E mutation reverses the increase in alkaline phosphatase toward normal.

A key enzyme in tissue calcification in vitro and in vivo is tissue non-specific alkaline phosphatase (TNAP).

Staining for alkaline phosphatase was performed using the Alkaline Phosphatase Detection Kit (Millipore). Assay of alkaline phosphatase was performed using the Quantitative Alkaline Phosphatase ES Characterization Kit (Millipore SCR066) according to the manufacturer's instructions. Briefly, cells were trypsinized and collected as 60,000 cells/reaction in p-nitrophenol phosphate substrate. Alkaline phosphatase was quantified by measuring p-nitrophenol produced, as gauged by absorption at 405 nm.

A model system for detecting tissue calcification employed calcific stimulation of confluent cells (Lazcano et al. Am. J. Clin. Pathol. 99:90-6 (1993); Rajamannan et al. Circulation 111:3296-301 (2005)). Cultures were treated with 0.1 μM dexamethasone, 50 μM ascorbic acid-2-phosphate and 10 mM β-glycerol phosphate in αMEM supplemented with 10% FBS and 1% penicillin/streptomycin for 21 days, replenishing the media every 4-5 days. At day 21, cells were washed with PBS and fixed in 10% formalin for 10 min. After washing with water, a solution of 2% Alizarin Red S, pH 4.2, was used to stain calcium phosphate crystals.

After 3 days of calcific stimulation, the fibroblasts of patient VI-4 of Family 1 stained abundantly for TNAP compared with control cells. Treatment with adenosine substantially reduced the amount of TNAP staining.

TNAP activity was also assayed in the lysates of fibroblasts grown in calcifying medium. Tables 5 and 6 show TNAP activity in fibroblasts from a control (Table 5) and from Patient VI-4 (Table 6) after incubation for 3 days in calcifying medium. Transduction with a control vector expressing beta-galactosidase had little effect on alkaline phosphatase activity, whereas transduction with a CD73-encoding vector reduced alkaline phosphatase activity significantly; incubation in 30 μM adenosine produced TNAP levels similar to those seen in control cells. Accordingly, as compared with control cells, cells from Patient VI-4 showed high levels of TNAP that were signifcantly reduced after transduction with CD73-encoded lentiviral vector or by adenosine treatment.

TABLE 5

| Treatment | TNAP (ng/6 × 104 cells) |
|---|---|
| No Treatment | 2.5 |
| Control Vector | 1 |
| CD73 | 0.6 |
| Adenosine | 1.5 |

TABLE 6

| Treatment | TNAP (ng/6 × 104 cells) |
|---|---|
| No Treatment | 7.2 |
| Control Vector | 5.7 |
| CD73 | 0.5 |
| Adenosine | 2 |

This example demonstrated that adenosine treatment of fibroblasts having the NT5E mutation in vitro reverses the increase in alkaline phosphatase toward normal.

### Example 7

This example demonstrates that adenosine treatment of fibroblasts having the NT5E mutation in vitro abrogates calcification.

The effects of interventions on calcium phosphate crystal formation in fibroblasts from a control and from Patient VI-4 were also studied. Control cells and cells from Patient VI-4 were cultured for 21 days in calcifying medium and stained for calcium with alizarin red S. After 21 days of calcific stimulation, the mutant fibroblasts produced abundant calcium phosphate crystals; normal fibroblasts produced none. Calcium phosphate crystal formation was prevented in cells transduced with a CD73-encoding lentiviral vector but not control vector expressing beta-galactosidase. Adenosine treatment (30 μM daily) largely abrogated the calcification process, and the noncompetitive alkaline phosphatase inhibitor levamisole (1 mM every fourth day) completely prevented calcification in the mutant cells.

### Example 8

This example demonstrates that cells with an NT5E mutation resulting in CD73 deficiency (ACDC) treated with exogenous AMP demonstrate no change/decrease in cAMP.

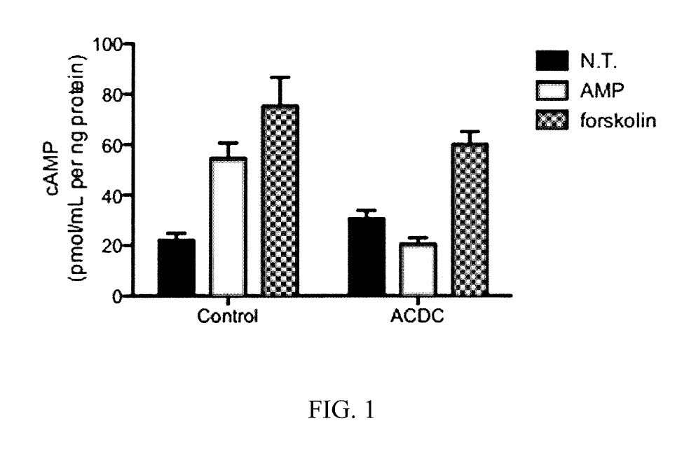

Control cells and cells with an NT5E mutation resulting in CD73 deficiency (arterial calcification due to deficiency of CD73 cells (ACDC) cells) were serum starved overnight then given 30 μM exogenous AMP or 2 μM forskolin (positive control) for 10 minutes and the levels or intracellular cAMP were assessed. The results are set forth in FIG. 1.

Control cells showed an increase in cAMP and ACDC cells showed no change/decrease in cAMP, suggesting that extracellular adenosine was binding to the A_{2A} or A_{2B} adenosine receptors (FIG. 1).

### Example 9

This example demonstrates that the A₃ agonist IB-MECA significantly inhibits TNAP activity in ACDC cells.

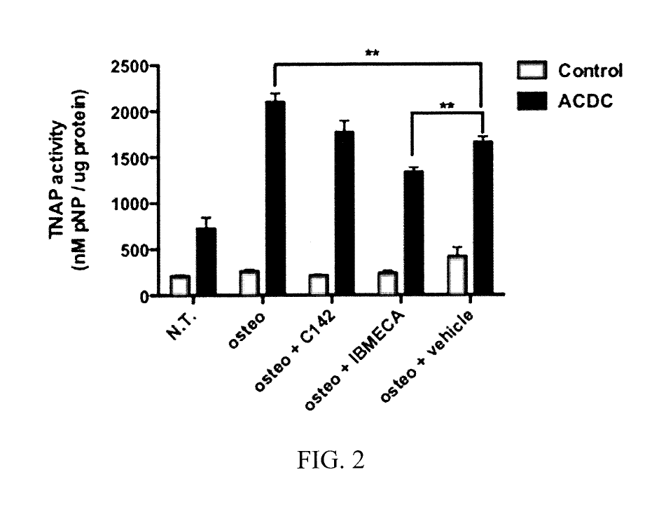

Control and ACDC cells were cultured for 10 days under growth conditions then fed with osteogenic media alone or with the A₁ agonist CCPA (C142) (2-chloro-N(6)-cyclopentyladenosine) hemihydrate (10 μM) or the A₃ agonist IB-MECA (10 μM) for 4 days and TNAP activity was assessed. The results are shown in FIG. 2.

The A₃ agonist IB-MECA significantly inhibited TNAP activity, suggesting a role for A₃ in inhibiting calcification (FIG. 2).

### Example 10

This example demonstrates that arterial and joint calcification is observed in patient II-1 of Family 3 of Example 1.

Radiographs showed extensive arterial calcification involving the distal femoral arteries and adjacent sections of the popliteal and posterior tibial arteries, as well as the external carotid arteries and the dorsalis pedis and posterior tibial arteries at the ankle. The internal carotids, aorta and coronary arteries were essentially devoid of calcification, as were the intracerebral vessels. The falx cerebri, the scar at the oligodendroglia excision, the thyroid and crycoid cartilages, and the costochondral cartilages showed premature calcifications; so did the periarticular connective tissue sheaths surrounding the joints of several interphalangeal and metacarpophalangeal joints. Coronary calcification was absent; however, the ligamentum arteriosum was calcified. Total body calcium was illustrated by non-contrast CT, showing calcification and arteriomegaly of the large arteries of the lower extremities. The lower extremities had an Agatston calcium score of 194485.

### Example 11

This example demonstrates that CD73 deficient fibroblasts from patient II-1 of Family 3 of Example 1 exhibit increased alkaline phosphatase staining and do not have an increase in collaged Iα1 mRNA expression 1-21 days after plating.

Alkaline phosphatase staining was performed using SIGMAFAST BCIP/NBT tablet (Sigma Aldrich), according to the manufacturer's instructions. Alkaline phosphatase activity was assayed with the StemTAG™ Alkaline Phosphatase Activity Assay Kit (Cell Biolabs, Inc.), using p-nitrophenol phosphate as substrate; the product, p-nitrophenol, was measured by absorption at 405 nm and normalized to μg protein per reaction.

RNA was isolated from cultured fibroblasts using the RNAeasy® kit (Qiagen) and 1 μg of RNA was used to generate cDNA using TaqMan Reverse Transcription reagents (Applied Biosystems). Expression of collagen Iα1 (COL1A1) mRNA was measured by quantitative realtime polymerase chain reaction (qPCR) on a Chroma4 Real Time PCR system (Bio-Rad) using iQ SYBR Green Supermix (Bio-Rad). Expression levels were calculated using the 2-ΔCt method, where the cycling threshold (Ct) of the candidate gene was compared to the Ct of 18s rRNA and expressed as a power of two (2(Ct-COL1A2-Ct-18s)). Primers: COL1A1 sense primer 5′-GCCGTGACCTCAAGATGTG-3′ (SEQ ID NO: 25), antisense primer 5′-GCCGAACCAGACATGCCTC-3′ (SEQ ID NO: 26) and 18S rRNA sense primer 5′-GTAACCCGTTGAACCCCATT-3′(SEQ ID NO: 27) antisense 5′-CCATCCAATCGGTAGTAGCG-3′ (SEQ ID NO: 28).

Fibroblasts cultured from the patient's skin biopsy exhibited increased alkaline phosphatase staining even without exposure to osteogenic medium. Treatment with osteogenic medium increased the amount of alkaline phosphatase staining in both normal and CD73-deficient fibroblasts, although the mutant cells were affected to a much greater extent. Quantification of alkaline phosphatase enzyme activity measurements confirmed the staining results (Table 9).

TABLE 9

| | Alkaline Phosphatase Activity (nM/pNP/μg protein) | |
|---|---|---|
| | No treatment | Osteogenic Medium |
| Control | 25 | 27 |
| Arterial Calcification | 130 | 160 |
| due to Deficiency of | | |
| CD73 cells (ACDC) | | |

Cells from patient and controls were isolated after 1, 7, 14, and 21 days in culture and illustrate that CD73-deficient cells do not have an increase in collagen Iα1 mRNA expression at these time-points (Table 10).

TABLE 10

| | Collagen Iα1 expression normalized to 18s | |
|---|---|---|
| Days post-plating | Control | ACDC |
| 1 | 1.00 | 1.25 |
| 7 | 1.00 | 1.00 |
| 14 | 0.60 | 0.65 |
| 21 | 0.25 | 0.30 |

### Example 12

This example demonstrates the histopathological similarity between Aterial Calcification due to Deficiency of CD73 (ACDC) and pseudoxanthoma elasticum (PXE).

Formalin fixed and paraffin-embedded sections of the femoral artery removed from patient II-1 of Family 3 of Example 1 during bypass surgery were surface decalcified and sectioned. The H&E stained sections performed at the University of San Francisco were obtained and imaged by oil immersion microscopy using Zeiss Plan NEOFLUOR 10×/0.30(DIC), Plan-APOCHROMAT 20×/0.8, and EC PLAN-NEOFLUAR 40×/1.3 oil objectives at 1.2 Mpixels per image (Zeiss Axiovert 200 M and Axiocam HRC, using Axiovision software, v 4.5). The UCSF H&E image was reconstructed from the 624 40× images using Photoshop CS4 by overlaying the individual images on a reconstructed 20× composite image that was reconstructed from 230 images formed by overlaying on a composite made from 36 10× images that were automatically assembled using photmerge. The final composite 2.35 GB image was cropped to 1.93 GB and stored as a PDF file. Individual sectional frames were assembled into a video tour of the master image to show the total image and the circumferential tract of the internal elastic lamina. The paraffin block was also sectioned at the NIH and three consecutive sections were stained. The first was stained with a routine H&E stain. The second and third sections were stained for elastin (Verhoeff s Technique of van Gieson staining), and Masson's Trichrome staining, respectively. The last two sections were stained using Dako Artisan kits (Dako North American Inc., Carpinteria, Calif. 93013). These three serial sections of the artery were imaged and the composites automatically assembled using Zeiss Axiovert 200 M with motorized scanning stage, Axiovision software v4.6, a Zeiss MRc5 camera, and the Plan-APOCHROMAT 20×/0.8 objective.

On histology, there was arteriomegaly with luminal stenosis due to a major expansion of the medial elements of the artery, and extensive calcifications. Von Kossa staining was strong for calcium deposition in the major lesion but was not definitive for the minor foci along the internal elastic lamina; the block had been decalcified. Close examination using Masson's trichrome, and elastin staining with the Verhoeff-van Gieson reagent, showed that the entire internal elastic lamina had varying degrees of duplication, along with fragmentation, loss of curvature, and staining of the broken ends, resembling the appearance of calcified fibers in pseudoxanthoma elasticum (PXE). A video survey at high magnification showed that the entire circumference was involved; the larger regions of calcification were apparent outgrowths of the elastic lamina. As each smaller nidus of calcification enlarges, differentiating cell types appear, including osteoblast-like cells inside of vacuolar spaces in the calcium-containing regions. Osteoclast-like cell clusters appeared to be remodeling these spaces. The van Gieson stain clearly shows a remaining degenerated fragment of the internal elastic lamina directly centered in the largest calcification focus of that section. Taken as a whole, the pathology of this disorder is consistent with changes in the internal elastic lamina inducing osteogenic activity that becomes circumferential and occludes the lumen.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing medial vascular or joint capsule calcification in a mammal in need thereof, comprising administering to the mammal an adenosine receptor agonist in an amount effective to treat or prevent the medial vascular or joint capsule calcification.

2. The method of claim 1, wherein treating or preventing medial vascular or joint capsule calcification in a mammal comprises treating or preventing a disorder selected from the group consisting of calciphylaxis, Monckeberg's medial sclerosis, arterial calcification due to deficiency of CD73 (ACDC), Ehlers Danlos syndrome, Marfan syndrome, Loewe Dietz syndrome, fibromuscular dysplasia, Kawasaki syndrome, pseudoxanthoma elasticum, and premature placental calcification.

3. The method of claim 1, comprising administering to the mammal an adenosine receptor agonist selected from the group consisting of an A₁ adenosine receptor agonist, an A₃ adenosine receptor agonist, an A_{2A} adenosine receptor agonist, and an A_{2B} receptor agonist, or a combination thereof.

4. The method of claim 1, wherein the mammal has a NT5E mutation.

5. The method of claim 1, wherein the pharmaceutically active agent is an A₁ adenosine receptor agonist.

6. The method of claim 5, wherein the A₁ adenosine receptor agonist is a compound of general formula (I): 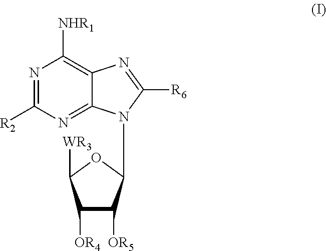
wherein R₁ represents a lower alkyl, substituted or unsubstituted cycloalkyl; a hydroxyl or hydroxyalkyl; a phenyl, anilide, or lower alkyl phenyl, all optionally substituted by one or more substituents —SOR^{c}, —SO₂R^{c}, —SO₃H, —SO₂NR^{a}R^{b}, —OR^{a}, —SR^{a}, —NHSO₂R^{c}, —NHCOR^{a}, —NR^{a}R^{b}, or —NHR^{a}CO₂R^{b}; wherein
R^{a} and R^{b} represent independently a hydrogen, lower alkyl, alkanoyl, amine, phenyl or naphthyl, the alkyl group optionally being substituted with a substituted or unsubstituted phenyl or phenoxy group; or when R₁ represents —NR^{a}R^{b}, said R^{a} and R^{b} form together with the nitrogen atom a 5- or 6-membered heterocyclic ring optionally containing a second heteroatom selected from oxygen or nitrogen, which second nitrogen heteroatom may optionally be further substituted by hydrogen or lower alkyl; or —NR^{a}R^{b} is a group of general formula (II) or (III): 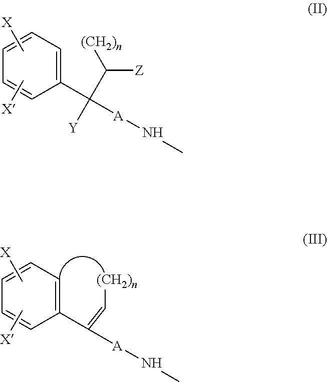
wherein n is an integer from 1 to 4; Z is hydrogen, lower alkyl or hydroxyl; Y is hydrogen, lower alkyl, or OR′ where R′ is hydrogen, lower alkyl or lower alkanoyl; A is a bond or a lower alkylene; X and X′ are each independently hydrogen, lower alkyl, lower alkoxy, hydroxy, lower alkanoyl, nitro, haloalkyl such as trifluoromethyl, halogen, amino, mono- or di-lower alkyl amino, or when X and X′ are taken together a methylenedioxy group;
R^{c} represents a lower alkyl; or
R₁ represents an epoxide substitutent of general formulae (IVa) or (IVb): 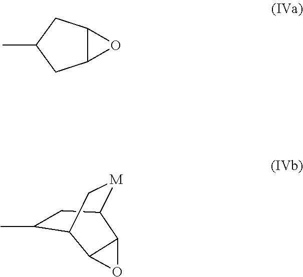
wherein M is a lower alkyl group;
R₂ represents hydrogen; halogen; substituted or unsubstituted lower alkyl or alkenyl group; lower haloalkyl or alkenyl; cyano; acetoamido; lower alkoxy; lower alkylamino; NR^{d}R^{e} where R^{d} and R^{e} are independently hydrogen, lower alkyl, phenyl or phenyl substituted by lower alkyl, lower alkoxy, halogen or haloalkyl or alkoxyl; —SR^{f} where R^{f} is hydrogen, lower alkyl, lower alkanoyl, benzoyl or phenyl;
W represents the group —OCH₂—, —NHCH₂—, —SCH₂—, or —NH(C═O)—;
R₃, R₄ and R₅ represent independently a hydrogen, lower alkyl or lower alkenyl, branched or unbranched C₁-C₁₂ alkanoyl, benzoyl or benzoyl substituted by lower alkyl, lower alkoxy, halogen, or R₄ and R₅ form together a 5-membered ring optionally substituted by a lower alkyl or alkenyl; R₃ further represents independently a phosphate, hydrogen or dihydrogen phosphate, or an alkali metal or ammonium or dialkali or diammonium salt thereof;
R₆ represents a hydrogen or halogen atom; or one of the substituents R₁ to R₆ is a sulfohydrocarbon radical of the formula R^{g}—SO₃—R^{h}—, wherein R^{g} represents a group selected from C₁-C₁₀ aliphatic, phenyl and lower alkyl substituted aromatic group which may be substituted or unsubstituted and R^{h} represents a monovalent cation, and the non-sulfur containing substituents being as defined above; or an isomer, diastereomer, pharmaceutically acceptable salt or solvate of said compound.

7. The method of claim 5, wherein the A₁ adenosine receptor agonist is selected from the group consisting of CPA, CCPA, S(−)-ENBA, ADAC, AMP579, NNC-21-0136, GR79236, CVT-510 (tecadenoson), CVT-2759, SDZ WAG 994, and selodenoson.

8. The method of claim 1, wherein the pharmaceutically active agent is an A₃ adenosine receptor agonist.

9. The method of claim 8, wherein the A₃ adenosine receptor agonist is a compound of general formula (V): 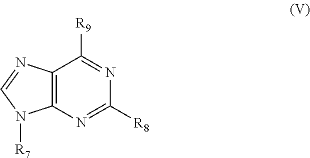
wherein R₇ is C₁-C₁₀ alkyl, C₁-C₁₀ hydroxyalkyl, C₁-C₁₀ carboxyalkyl or C₁-C₁₀ cyanoalkyl or a group of the following general formula (VI): 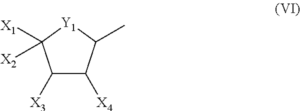
in which:
Y₁ is an oxygen or sulfur atom or CH₂;
X₁ is H, C₁-C₁₀ alkyl, R^{100a}R^{100b}NC(═O) or HOR^{100c}—, wherein R^{100a} and R^{100b} may be the same or different and are selected from the group consisting of hydrogen, C₁-C₁₀ alkyl, amino, C₁-C₁₀ haloalkyl, C₁-C₁₀ aminoalkyl, t-butoxycarbonyl-aminoalkyl, and C₃-C₁₀ cycloalkyl or are joined together to form a heterocyclic ring containing two to five carbon atoms, and R^{100c} is selected from the group consisting of C₁-C₁₀ alkyl, amino, C₁-C₁₀ haloalkyl, C₁-C₁₀ aminoalkyl, C₁-C₁₀ butyloxycarbonyl (BOC)-aminoalkyl, and C₃-C₁₀ cycloalkyl;
X₂ is H, hydroxyl, C₁-C₁₀ alkylamino, C₁-C₁₀ alkylamido or C₁-C₁₀ hydroxyalkyl;
X₃ and X₄ each independently are hydrogen, hydroxyl, amino, amido, azido, halo, alkyl, alkoxy, carboxy, nitrilo, nitro, trifluoro, aryl, alkaryl, mercapto, thioester, thioether, —OCOPh, —OC(═S)OPh or both X₃ and X₄ are oxygen connected to >C═S to form a 5-membered ring, or X₂ and X₃ form the ring of formula (VII): 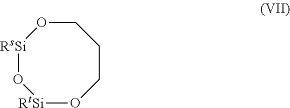
where R^{s} and R^{t} are independently C₁-C₁₀ alkyl;
R₈ is selected from the group consisting of hydrogen, halo, C₁-C₁₀ alkylether, amino, hydrazido, C₁-C₁₀ alkylamino, C₁-C₁₀ alkoxy, C₁-C₁₀ thioalkoxy, pyridylthio, C₂-C₁₀ alkenyl, C₂-C₁₀ alkynyl, mercapto, and C₁-C₁₀ alkylthio; and
R₉ is a —NR₁₀R₁₁ group with R₁₀ being hydrogen, alkyl, substituted alkyl or aryl-NH—C(Z₁)—, with Z₁ being O, S or NR^{100a}, and, when R₁₀ is hydrogen, R₁₁ being selected from the group consisting of R- and S-1-phenylethyl, benzyl, phenylethyl or anilide groups, each said group being unsubstituted or substituted in one or more positions with a substituent selected from the group consisting of C₁-C₁₀ alkyl, amino, halo, C₁-C₁₀ haloalkyl, nitro, hydroxyl, acetamido, C₁-C₁₀ alkoxy, and sulfonic acid or a salt thereof; or R₁₁ being benzodioxanemethyl, furfuryl, L-propylalanylaminobenzyl, β-alanylaminobenzyl, t-BOC-β-alanylaminobenzyl, phenylamino, carbamoyl, phenoxy or C₁-C₁₀ cycloalkyl; or R₁₁ being a group of the following formula (VIII): 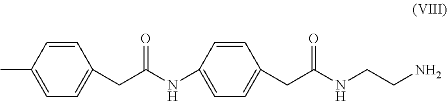 [SMILES: Cc1ccc(CC(=O)Nc2ccc(CC(=O)NCCN)cc2)cc1]
or, when R₁₀ is alkyl, substituted alkyl, or aryl-NH—C(Z₁)—, then R₁₁ being selected from the group consisting of substituted or unsubstituted heteroaryl-NR^{100a}—C(Z₁), heteroaryl-C(Z₁)—, alkaryl-NR^{100a}—C(Z₁)—, alkaryl-C(Z₁)—, aryl-NR—C(Z₁)— and aryl-C(Z₁); or an isomer, diastereomer, pharmaceutically, acceptable salt or solvate of said compound.

10. The method of claim 8, wherein the A₃ adenosine receptor agonist is selected from the group consisting of IB-MECA, C1-IB-MECA, LJ568, CP-608039, MRS3558, and MRS1898.

11. The method of claim 1, wherein the pharmaceutically active agent is an A_{2A} adenosine receptor agonist.

12. The method of claim 11, wherein the A_{2A} adenosine receptor agonist is a compound of general formula (IX): 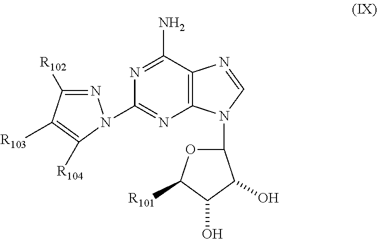
wherein R₁₀₁═CH₂OH or —CONR₁₀₅R₁₀₆;
R₁₀₃ is independently selected from the group consisting of C₁₋₁₅ alkyl, halo, NO₂, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂, CONR₁₀₇R₁₀₈, C₂₋₁₅ alkenyl, C₂₋₁₅ alkynyl, heterocyclyl, aryl, and heteroaryl, wherein the alkyl, alkenyl, alkynyl, aryl, heterocyclyl and heteroaryl substituents are optionally substituted with from 1 to 3 substituents independently selected from the group consisting of halo, alkyl, NO₂, heterocyclyl, aryl, heteroaryl, CF₃, CN, OR²⁰, SR²⁰, N(R)²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein the optionally substituted heteroaryl, aryl, and heterocyclyl substituents are optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, or OR²⁰;
R₁₀₅ and R₁₀₆ are each individually selected from H, and C₁-C₁₅ alkyl that is optionally substituted with from 1 to 2 substituents independently selected from the group of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ wherein each optionally substituted heteroaryl, aryl, and heterocyclyl substituent is optionally substituted with halo, NO₂, alkyl, CF₃, amino, monoalkylamino, dialkylamino, alkylamide, arylamide, heteroarylamide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, and OR²⁰;
R₁₀₇ is selected from the group consisting of hydrogen, C₁₋₁₅ alkyl, C₂₋₁₅ alkenyl, C₂₋₁₅ alkynyl, heterocyclyl, aryl and heteroaryl, wherein the alkyl, alkenyl, alkynyl, aryl, heterocyclyl and heteroaryl substituents are optionally substituted with from 1 to 3 substituents independently selected from the group of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰ and OCON(R²⁰)₂ and wherein each optionally substituted heteroaryl, aryl and heterocyclyl substituent is optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N (R²⁰)₂, SR²⁰, S(O)R²², SO₂R²², SO₂N(R²⁰)₂, CN, and OR²⁰;
R₁₀₈ is selected from the group consisting of hydrogen, C₁₋₁₅ alkyl, C₂₋₁₅ alkenyl, C₂₋₁₅ alkynyl, heterocyclyl, aryl, and heteroaryl, wherein the alkyl, alkenyl, alkynyl, aryl, heterocyclyl, and heteroaryl substituents are optionally substituted with from 1 to 3 substituents independently selected from the group consisting of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein each optionally substituted heteroaryl, aryl, and heterocyclyl substituent is optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, and OR²⁰;
R²⁰ is selected from the group consisting of H, C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl substituents are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl;
R²² is selected from the group consisting of C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl substituents are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl; and
wherein R₁₀₂ and R₁₀₄ are selected from the group consisting of H, C₁₋₆ alkyl and aryl, wherein the alkyl and aryl substituents are optionally substituted with halo, CN, CF₃, OR²⁰ and N(R²⁰)₂ with the proviso that when R₁₀₂ is not hydrogen then R₁₀₄ is hydrogen, and when R₁₀₄ is not hydrogen then R₁₀₂ is hydrogen.

13. The method of claim 11, wherein the A_{2A} adenosine receptor agonist is selected from the group consisting of NECA; CGS21680; MRE-0094; DPMA; Glaxo compound; binodenoson; apadenoson; ATL-313; and regadenoson.

14. The method of claim 1, wherein the pharmaceutically active agent is an A_{2B} adenosine receptor agonist.

15. The method of claim 14, wherein the A_{2B} adenosine receptor agonist is a compound of general formula (X): 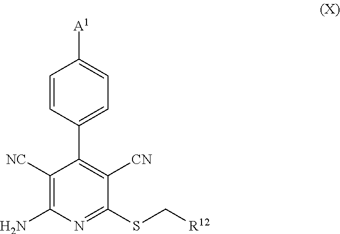
wherein
A¹ represents —O—R¹³ or —NH—C(═O)—R¹⁴;
R¹² represents CH₁₂—C(═O)—NH₂, pyridyl or thiazolyl;
R¹³ represents hydrogen or (C₃-C₆)-cycloalkylmethyl, and
R¹⁴ represents (C₁-C₄)-alkyl, (C₁-C₄)-alkoxy, mono- or di-(C₁-C₄)-alkylamino.

16. The method of claim 14, wherein the A_{2B} adenosine receptor agonist is LUF5835 or Bay-60-658.

17-18. (canceled)

19. A method of detecting or diagnosing a vascular or joint capsule calcification disorder comprising
(a) obtaining a nucleic acid sample from a mammal;
(b) obtaining a NT5E coding sequence from the nucleic acid sample; and
(c) comparing the NT5E coding sequence of the nucleic acid in the sample to a NT5E coding sequence of a negative control and obtaining a difference, wherein the difference is due to a mutation in the NT5E coding sequence of the nucleic acid in the sample.

20. The method of claim 1, wherein the vascular or joint capsule calcification disorder is due to CD73 deficiency.

21. The method of claim 4, wherein the mutation is (a) a missense mutation, (b) a nonsense mutation, (c) an insertion mutation, or (d) a deletion mutation.

22. The method of claim 4, wherein the mutation is located in one or more exons of NT5E selected from the group consisting of Exon 3, Exon 5, and Exon 9.

23. The method of claim 4, wherein the mutation comprises a substitution or insertion of a nucleotide residue of non-mutant human NT5E selected from the group consisting of (a) nucleotide residue 100 of Exon 3; (b) nucleotide residue 124 of Exon 5; and (c) nucleotide residue 48 of Exon 9.

24. The method of claim 19, further comprising detecting a nucleic acid sequence selected from the group consisting of SEQ ID NOs: 3, 5, and 7 or a combination thereof.

25. A nucleic acid comprising a mutation in one or more exons of human NT5E selected from the group consisting of Exon 3, Exon 5, and Exon 9.

26. The nucleic acid of claim 25, wherein the mutation comprises a substitution or insertion of a nucleotide residue of non-mutant human NT5E selected from the group consisting of (a) nucleotide residue 100 of Exon 3; (b) nucleotide residue 124 of Exon 5; and (c) nucleotide residue 48 of Exon 9.

27. The nucleic acid of claim 26, wherein the nucleic acid comprises a nucleotide sequence selected from the group consisting of SEQ ID NO:s 3, 5, and 7.

28. A polypeptide encoded by a nucleic acid comprising a mutation in Exon 5 or 9 of human NT5E.

29. A polypeptide encoded by a nucleic acid comprising a mutation comprising a substitution or insertion of a nucleotide residue of non-mutant human NT5E selected from the group consisting of (a) nucleotide residue 124 of Exon 5 and (b) nucleotide residue 48 of Exon 9.

30. A polypeptide encoded by a nucleic acid comprising SEQ ID NO: 5 or 7.

31. The polypeptide of claim 28 comprising the amino acid sequence of any one of SEQ ID NOs: 13-14.

32. A polypeptide consisting of SEQ ID NO: 12.

33. A vector comprising the nucleic acid of claim 25.

34. A recombinant cell comprising the vector of claim 33.

35. A method of determining reduced activity or expression level of CD73 protein comprising:
(a) determining the activity or expression level of CD73 protein of a biological sample from a mammal, and
(b) comparing the activity or expression level of the CD73 protein in the mammal with a negative control.

36. An antibody, or an antigen binding portion thereof, that specifically binds to the polypeptide of claim 28 but does not bind to a polypeptide encoded by non-mutant NT5E.

37. A method of detennining an NT5E mutation comprising:
(a) contacting a biological sample from a mammal with an antibody that binds to a polypeptide encoded by a nucleic acid comprising a mutation in Exon 5 or 9 of human NT5E but does not bind to a polypeptide encoded by non-mutant NT5E; and
(b) detecting the binding of the antibody to the polypeptide encoded by a nucleic acid comprising a mutation in Exon 5 or 9 of human NT5E.


SEQUENCE LISTING (28 sequences)

SEQ ID NO: 1 - DNA, 1725 bp, Homo sapiens
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatag

SEQ ID NO: 2 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 3 - DNA, 189 bp, Homo sapiens
ggacaaatttagtgtttgaagatgaaatcactgcattacaacctgaagtagataagttaaaaactctaaatgtgaacaaaattattgcactgggacattagggttttgaaatggataaactcatcgctcagaaagtgaggggtgtggacgtcgtggtgggaggacactccaacacatttctttacacag

SEQ ID NO: 4 - DNA, 189 bp, Homo sapiens
ggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacag

SEQ ID NO: 5 - DNA, 155 bp, Homo sapiens
atccaagcataaaagcagacattaacaaatggaggataaaattggataattattctacccaggaattagggaaaacaattgtctatctggatggctcctctcaatcatgccgctttagagaatacaacatgggcaacctgatttgtgatgcaatg

SEQ ID NO: 6 - DNA, 155 bp, Homo sapiens
atccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatg

SEQ ID NO: 7 - DNA, 165 bp, Homo sapiens
gtgaccaagatatcaacgtggtttctacatatatctccaaaatgaaaagtaatttatccagcagttgaaggtcggatcaagttttccacaggaagtcactgccatggaagcttttctttaatatttctttcactttgggcagtgatctttgttttataccaatag

SEQ ID NO: 8 - DNA, 164 bp, Homo sapiens
gtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatag

SEQ ID NO: 9 - DNA, 1725 bp, Homo sapiens
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattagggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatag

SEQ ID NO: 10 - DNA, 1725 bp, Homo sapiens
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatacaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatag

SEQ ID NO: 11 - DNA, 1726 bp, Homo sapiens
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaaagtaatttatccagcagttgaaggtcggatcaagttttccacaggaagtcactgccatggaagcttttctttaatatttctttcactttgggcagtgatctttgttttataccaatag

SEQ ID NO: 12 - protein, 220 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGH

SEQ ID NO: 13 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFREYNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 14 - protein, 542 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKSNLSSS

SEQ ID NO: 15 - DNA, 19 bp, Artificial Sequence
  Synthetic
gggcggaaggttcctgtag

SEQ ID NO: 16 - DNA, 21 bp, Artificial Sequence
  Synthetic
gaggagccatccagatagaca

SEQ ID NO: 17 - DNA, 20 bp, Artificial Sequence
  Synthetic
gtaacccgttgaaccccatt

SEQ ID NO: 18 - DNA, 20 bp, Artificial Sequence
  Synthetic
ccatccaatcggtagtagcg

SEQ ID NO: 19 - DNA, 45 bp, Artificial Sequence
  Synthetic
tgtgaacaaaattattgcactgggacattagggttttgaaatgga

SEQ ID NO: 20 - DNA, 45 bp, Artificial Sequence
  Synthetic
tccatttcaaaaccctaatgtcccagtgcaataattttgttcaca

SEQ ID NO: 21 - DNA, 34 bp, Artificial Sequence
  Synthetic
catgccgctttagagaatacaacatgggcaacct

SEQ ID NO: 22 - DNA, 35 bp, Artificial Sequence
  Synthetic
caggttgcccatgttgtattctctaaagcggcatg

SEQ ID NO: 23 - DNA, 49 bp, Artificial Sequence
  Synthetic
ctacatatatctccaaaatgaaaagtaatttatccagcagttgaaggtc

SEQ ID NO: 24 - DNA, 49 bp, Artificial Sequence
  Synthetic
gaccttcaactgctggataaattacttttcattttggagatatatgtag

SEQ ID NO: 25 - DNA, 19 bp, Artificial Sequence
  Synthetic
gccgtgacctcaagatgtg

SEQ ID NO: 26 - DNA, 19 bp, Artificial Sequence
  Synthetic
gccgaaccagacatgcctc

SEQ ID NO: 27 - DNA, 20 bp, Artificial Sequence
  Synthetic
gtaacccgttgaaccccatt

SEQ ID NO: 28 - DNA, 20 bp, Artificial Sequence
  Synthetic
ccatccaatcggtagtagcg